(19) United States
(12) Patent Application Publication
(10) Pub. No.: US 2010/0041860 A1
(43) Pub. Date: Feb. 18, 2010

(54) POLYIMIDE, DIAMINE COMPOUND AND METHOD FOR PRODUCING THE SAME

(75) Inventors: Hiroaki Yamaguchi, Ube-Shi (JP); Shuichi Maeda, Ube-Shi (JP); Nobuharu Hisano, Ube-Shi (JP); Shinsuke Yabunaka, Ube-shi (JP); Kiyotaka Yoshii, Ube-Shi (JP); Masayoshi Ohue, Ube-Shi (JP); Akio Matsushita, Ube-Shi (JP); Yasuhiro Kawachi, Ube-Shi (JP)

Correspondence Address: KNOBBE MARTENS OLSON & BEAR LLP, 2040 MAIN STREET, FOURTEENTH FLOOR, IRVINE, CA, 92614, US

(73) Assignee: UBE INDUSTRIES, LTD., UBE-SHI, YAMAGUCHI (JP)

(21) Appl. No.: 12/514,437

(22) PCT Filed: Nov. 12, 2007
(86) PCT No.: PCT/JP2007/071934

§ 371 Date: Jun. 12, 2009

(30) Foreign Application Priority Data
Nov. 10, 2006 (JP) 2006-305196
Mar. 30, 2007 (JP) 2007-090482
Mar. 30, 2007 (JP) 2007-093731
Mar. 30, 2007 (JP) 2007-093732

(51) Int. Cl. C08G 69/26; C07C 229/52

(52) U.S. Cl. 528/353; 562/457

(57) ABSTRACT

A polyimide obtained by reacting a tetracarboxylic acid component with a diamine component containing a diamine compound represented by the following general formula (1):

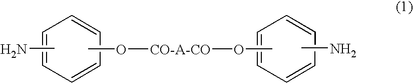

wherein, A represents a biphenylene group which may be substituted with an alkyl group having up to 4 carbon atoms.

### TECHNICAL FIELD

The present invention relates to a novel polyimide. More particularly, the invention relates to a polyimide resin obtained from a tetracarboxylic acid component and a diamine component containing a novel diamine compound. Further, the invention relates to a novel diamine compound which is particularly suitable for the production of a polyimide.

### BACKGROUND ART

Since polyimide films are excellent in thermal and electric properties, polyimide films have been widely used for electronic devices such as flexible printed circuit boards, tapes for TAB (Tape Automated Bonding) and the like. In particular, there has been known that a polyimide having a high elastic modulus with a low linear expansion coefficient is obtained from 3,3′,4,4′-biphenyltetracarboxylic dianhydride and p-phenylenediamine respectively as a tetracarboxylic acid component and a diamine component.

In the use of a flexible printed circuit board, TAB or the like, dimensional stability of a polyimide to be used has been demanded. For example, when the difference between the thermal expansion coefficient of a film and that of copper becomes big, curling occurs and processing accuracy is lowered so that it is difficult to achieve accurate mounting of electronic components. Further, since the wiring pattern is formed by etching a laminated copper foil, there is a problem in that processing accuracy and mounting accuracy of a wiring pattern are lowered due to expansion by water absorption and shrinkage by drying. In view of these problems, a polyimide film having low water absorption percentage and low hygroscopic expansion coefficient have been demanded in addition to a thermal expansion coefficient.

Japanese Laid-open Patent Publication No. H11-199668 (Patent Document 1) discloses a polyimide structure based on a diamine component comprising a diamine compound represented by H₂N—Ph-OCO—X—CO(O-Ph-NH₂ (herein, X is a phenylene group) and a tetracarboxylic acid component as a polyimide exhibiting low water absorption and low hygroscopic expansion property. However, according to the review made by the present inventors, the water absorption percentage and the hygroscopic expansion coefficient are not yet sufficient when X represents a phenylene group. Furthermore, since vibration or bending is repeatedly applied in the use of a flexible printed circuit board, elongation at break has been required in addition to breaking strength. Whereas, the polyimide based on the above diamine compound have shown greatly reduced elongation at break. In addition, in Patent Document 1, other groups are exemplified as X, but it fails to demonstrate the effectiveness of the polyimide obtained from the diamine component in which X is a group other than a phenylene group.

Patent Document 1: Japanese Laid-open Patent Publication No. H11-199668

### DISCLOSURE OF THE INVENTION

### Problems to be Solved by the Invention

An object of the present invention is to provide a polyimide material having a low water absorption percentage and a low hygroscopic expansion coefficient. In particular, an object of the present invention is to provide a polyimide material in which a water absorption percentage and a hygroscopic expansion coefficient are reduced without greatly lowering elongation at break.

Further, an object of another aspect of the present invention is to provide a novel diamine compound to be used as a raw material for the production of a polyimide having such properties.

### Means for Solving the Problems

The present invention relates to the following matters.

1. A polyimide obtained by reacting a tetracarboxylic acid component with a diamine component containing a diamine compound represented by the following general formula (1):

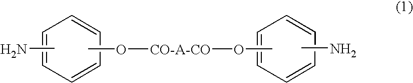

wherein, A represents a biphenylene group which may be substituted with an alkyl group having up to 4 carbon atoms.

2. The polyimide according to the above item 1, wherein the tetracarboxylic acid component comprises 3,3′,4,4′-biphenyltetracarboxylic dianhydride in an amount of 10 mol % or more of all tetracarboxylic acid components.

3. The polyimide according to the above item 1 or 2, wherein the diamine compound represented by the above general formula (1) comprises a compound represented by the following formula (1a):

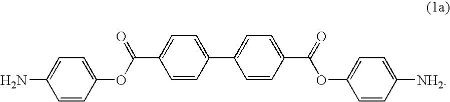

4. A polyimide film comprising the polyimide according to any one of the above items 1 to 3.

5. A diamine compound represented by the general formula (1):

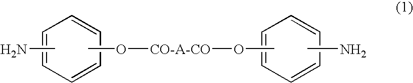

wherein, A represents a biphenylene group which may be substituted with an alkyl group having up to 4 carbon atoms.

6. Biphenyl-4,4′-dicarboxylic acid bis(4-aminophenyl)ester represented by the following formula (1a):

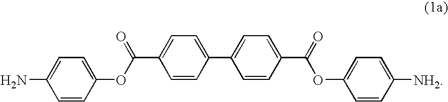

7. A method for producing the diamine compound represented by the general formula (1) according to the above item 5, comprising steps of

reacting a biphenyl dicarbonyl halide derivative represented by the general formula (2):

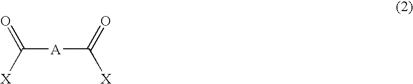

wherein, A represents a biphenylene group which may be substituted

with an alkyl group having up to 4 carbon atoms; and X represents a halogen atom,

with nitrophenol in the presence of a base, whereby producing biphenyl-dicarboxylic acid bis(nitrophenyl)ester represented by the general formula (3):

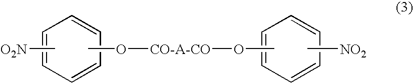

and

reducing biphenyl-dicarboxylic acid bis(nitrophenyl)ester represented by the above general formula (3).

8. A method for producing the diamine compound represented by the general formula (1) according to the above item 5, comprising:

reacting a biphenyl carbonyl derivative represented by the general formula (21):

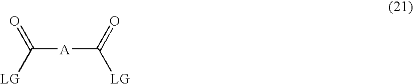

wherein, A has the same meaning as defined above; and LG is a

leaving group which can be exchanged with an aminophenoxy group, with aminophenol in the presence of a base.

9. The method for producing the diamine compound according to the above item 8, wherein the above general formula (21) is a biphenyl-dicarboxylic acid bis(aryl)ester compound represented by the following general formula (22):

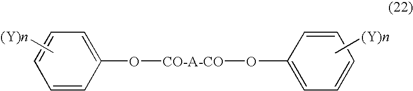

wherein, A has the same meaning as defined above; Y represents a halogen atom, a nitro group, a trifluoromethyl group, a cyano group or an acetyl group; and n represents an integer of 0 to 3.

10. The method for producing the diamine compound according to the above item 9, wherein the biphenyl-4,4′-dicarboxylic acid bis(aryl)ester compound represented by the above formula (22) is obtained by reacting the biphenyldicarbonyl halide derivative represented by the general formula (2):

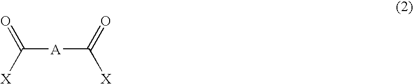

wherein, A represents a biphenylene group which may be substituted

with an alkyl group having up to 4 carbon atoms; and X represents a halogen atom,

a hydroxyaryl compound represented by the general formula (23):

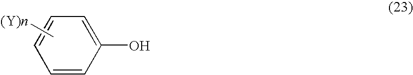

wherein, Y and n have the same meaning as defined above, and a base.

11. The method for producing the diamine compound according to the above item 9, wherein the reaction is carried out without removing the generated hydroxyaryl compound from the reaction solution.

12. The method for producing the diamine compound according to the above item 9, wherein the reaction is carried out while removing the generated hydroxyaryl compound from the reaction solution.

13. The method for producing the diamine compound according to the above item 9, wherein a substitution position in the aryl moiety of the biphenyl-dicarboxylic acid bis(aryl)ester compound of the above general formula (22) is at least one substitution position selected from the group consisting of the 2 position, the 4 position and the 6 position.

14. The method for producing the diamine compound according to the above item 9, wherein Y is a chlorine atom.

15. A biphenyl-dicarboxylic acid bis(aryl)ester compound represented by the following general formula (22):

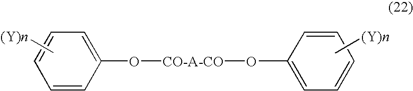

wherein, A represents a biphenylene group which may be substituted with an alkyl group having up to 4 carbon atoms; Y represents a halogen atom, a nitro group, a trifluoromethyl group, a cyano group or an acetyl group; and n represents an integer of 0 to 3;

with the proviso that biphenyl-4,4′-dicarboxylic acid diphenyl ester, biphenyl-4,4′-dicarboxylic acid bis(2-chlorophenyl)ester and biphenyl-4,4′-dicarboxylic acid bis(2-nitrophenyl)ester are excluded.

16. The biphenyl-dicarboxylic acid bis(aryl)ester compound according to the above item 15, wherein A represents a 4,4′-biphenylene group.

17. The method for producing the diamine compound according to the above item 8, wherein the above general formula (21) is a biphenyl carbamide compound represented by the general formula (32):

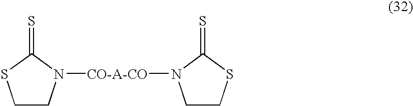

wherein, A has the same meaning as defined above.

18. The method for producing the diamine compound according to the above item 17, wherein the biphenyl carbamide compound represented by the above formula (32) is obtained by reacting the biphenyldicarbonyl halide derivative represented by the general formula (2):

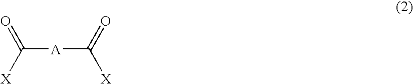

wherein, A represents a biphenylene group which may be substituted

with an alkyl group having up to 4 carbon atoms; and X represents a halogen atom,

2-thiazoline-2-thiol and a base.

19. A biphenyl carbamide compound represented by the general formula (32):

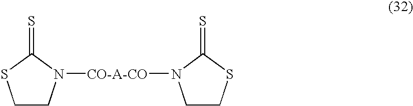

wherein, A represents a biphenylene group which may be substituted with an alkyl group having up to 4 carbon atoms.

20. The biphenyl carbamide compound according to the above item 19, wherein A represents a 4,4′-biphenylene group.

### Effect of the Invention

The polyimide of the present invention is excellent in heat resistance, has a low water absorption percentage and a low linear hygroscopic expansion coefficient, and is excellent in dimensional stability In particular, since the compound of the above formula (1) is contained in the raw material diamine component, a polyimide having a low water absorption percentage and a low linear hygroscopic expansion coefficient is easily obtained without greatly lowering elongation at break. Accordingly, the polyimide of the present invention can be suitably used for a film for TAB, a board for an electronic component, a wiring board or the like.

Further, according to the present invention, it is possible to provide a novel diamine compound to be a raw material for the production of a polyimide having excellent properties and its production method.

### BEST MODE FOR CARRYING OUT THE INVENTION

The present invention relates to a polyimide obtained by reacting a diamine component containing a diamine compound of the formula (1) with a tetracarboxylic acid component.

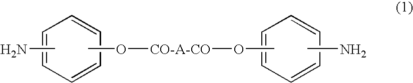

In the formula (1), A is a biphenylene group which may have substituent(s) and preferably a 4,4′-biphenylene group represented by the formula (A1):

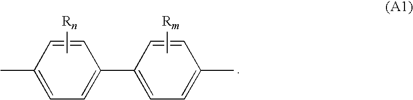

Herein, n and m each represents the number of substituent R on each ring and each independently represents 0, 1, 2, 3 or 4, and when both n and m are 0, the compound of the formula (A1) represents an unsubstituted 4,4′-biphenylene group. R represents an alkyl group having up to 4 carbon atoms, and preferably methyl group, ethyl group, propyl group or the like. In case R occurs more than once in the formula (A1), each R, independently one another, has the same meaning as defined above. A is preferably a biphenylene group represented by the formula (A2), (A3), (A4) or (A5), and most preferably a group represented by the formula (A2),

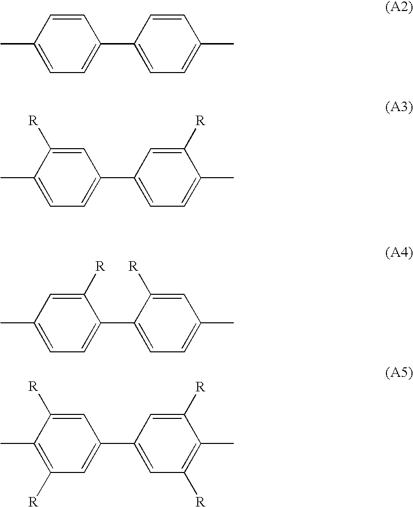

wherein, R has the same meaning as defined above.

A terminal —NH₂ group of the compound of the formula (1) is bonded to a phenylene group at the ortho position, the meta position or the para position relative to an —O— group. Preferably, the terminal —NH₂ group of the compound of the formula (1) is bonded to the phenylene group at the para position relative to the —O— group.

The diamine component used for the production of the polyimide of the present invention contains the diamine compound of the formula (1), whereby the water absorption percentage of the polyimide can be lowered. The diamine compound of the formula (1) may be contained, depending on the embodiments, in the ratio of 5 mol % or more, 10 mol % or more, preferably 30 mol % or more, more preferably 50 mol % or more, further preferably 60 mol % or more, and further preferably 70 mol % or more, in 100 mol % of the diamine compound. In specific aspects, the content may be 100 mol %.

Furthermore, as exemplified in APBP unit to be described below, weight % of the constituent unit composed of the acid dianhydride component and the diamine component of the formula (1) may be contained, depending on the embodiments, in the ratio of 5 weight % or more, 15 weight % or more, preferably 40 weight % or more, more preferably 50 weight % or more, 60 weight % or more, further preferably 70 weight % or more, and 80 weight % or more, in 100 weight % of the constituent unit. In specific aspects, it may be 100 weight %.

The diamine component may contain, in addition to the diamine compound of the formula (1), one or two or more diamine compounds other than the diamine compound of the formula (1). The examples of the diamine compound include p-phenylenediamine, m-phenylenediamine, 4,4′-diaminodiphenylprop ane, 4,4-diaminodiphenylmethane, benzidine, 3,3′-dichlorobenzidine, 4,4′-diaminodiphenylsulfide, 3,3′-diaminodiphenylsulfone, 4,4′-diaminodiphenylsulfone, 4,4′-oxydianiline, 3,3′-oxydianiline, 3,4′-oxydianiline, 1,5-diaminonaphthalene, 4,4′-diaminodiphenyldiethylsilane, 4,4′-diaminodiphenylsilane, 4,4′-diaminodiphenylethyl phosphine oxide, 1,4-diaminobenzene(p-phenylenediamine), 1,4-diaminobenzene(p-phenylenediamine), bis{4-(4-aminophenoxy)phenyl}sulfone, bis{4-(3-aminophenoxy)phenyl}sulfone, 4,4′-bis(4-aminophenoxy)biphenyl, 4,4′-bis(3-aminophenoxy)biphenyl, 1,3-bis(3-aminophenoxy)benzene, 1,3-bis(4-aminophenoxy)benzene, 1,4-bis(4-aminophenoxy)benzene, 1,4-bis(3-aminophenoxy)benzene, 3,3′-diaminobenzophenone, 4,4′-diaminobenzophenone and analogues thereof.

Furthermore, The examples of the diamine compound other than the diamine compound of the formula (1) include 3,3′,5,5′-tetramethyl-4,4′-diaminobiphenyl, 3,3′,5,5′-tetramethyl-4,4′-diaminodiphenyl ether, 3,3′,5,5′-tetramethyl-4,4′-diaminodiphenylmethane, 3,3′,5,5′-tetraethyl-4,4′-diaminobiphenyl, 3,3′,5,5′-tetraethyl-4,4′-diaminodiphenyl ether, 3,3′,5,5′-tetraethyl-4,4′-diaminodiphenylmethane, 4,4-methylene-bis(2,6-diisopropylaniline), 3,3′-dicarboxy-4,4′-diamino-5,5′-dimethyldiphenylmethane, 3,3′-dimethyl-4,4′-diaminobiphenyl, 2,2′-dimethyl-4,4′-diaminobiphenyl, 3,3′-diethyl-4,4′-diaminobiphenyl, 3,3′-dihydroxy-4,4′-diaminobiphenyl, 3,3′-dicarboxy-4,4′-diaminobiphenyl, 3,3′-dimethoxy-4,4′-diaminobiphenyl, 3,3′-dimethyl-4,4′-diaminodiphenyl ether, 3,3′-diethyl-4,4′-diaminodiphenyl ether, 3,3′-dihydroxy-4,4′-diaminodiphenyl ether, 3,3′-dicarboxy-4,4′-diaminodiphenyl ether, 3,3′-dimethoxy-4,4′-diaminodiphenyl ether, 3,3′-dimethyl-4,4′-diaminodiphenylmethane, 3,3′-diethyl-4,4′-diaminodiphenylmethane, 3,3′-dihydroxy-4,4′-diaminodiphenylmethane, 3,3′-dicarboxy-4,4′-diaminodiphenylmethane, 3,3′-dimethoxy-4,4′-diaminodiphenylmethane and the like.

Of these diamine compounds, as the diamine compound which is preferably used together with the diamine compound of the formula (1), the examples thereof include p-phenylenediamine, 4,4-diaminodiphenylmethane, 4,4′-diaminodiphenylsulfide, 3,3′-diaminodiphenylsulfone, 4,4′-diaminodiphenylsulfone, 4,4′-oxydianiline, 3,3′-dimethyl-4,4′-diaminobiphenyl, 2,2′-dimethyl-4,4′-diaminobiphenyl, 1,3-bis(3-aminophenoxy)benzene, 1,3-bis(4-aminophenoxy)benzene, 1,4-bis(4-aminophenoxy)benzene, 1,4-bis(3-aminophenoxy)benzene, 3,3′-diaminobenzophenone and 4,4′-diaminobenzophenone. Further preferably used are p-phenylenediamine and 4,4′-oxydianiline.

As the tetracarboxylic acid component, known tetracarboxylic anhydrides can be used. The examples of the tetracarboxylic dianhydride include aromatic tetracarboxylic dianhydrides such as 3,3′,4,4′-biphenyltetracarboxylic dianhydride, pyromellitic dianhydride, 2,3,3′,4′-biphenyltetracarboxylic dianhydride, 3,3′,4,4′-benzophenonetetracarboxylic dianhydride, 2,2-bis(3,4-dicarboxyphenyl)propane dianhydride, bis(3,4-dicarboxyphenyl)methane dianhydride, bis(3,4-dicarboxyphenyl)ether dianhydride, bis(3,4-dicarboxyphenyl)sulfone dianhydride, bis(3,4-dicarboxyphenyl)sulfide dianhydride, p-phenylenebis(trimellitic monoester anhydride), ethylenebis(trimellitic monoester anhydride), bisphenol A bis(trimellitic monoester anhydride), 2,2-bis(3,4-dicarboxyphenyl)-1,1,1,3,3,3-hexafluoropropane dianhydride, 2,2-bis(2,3-dicarboxyphenyl)-1,1,1,3,3,3-hexafluoropropane dianhydride, 1,2,5,6-naphthalenetetracarboxylic dianhydride, 2,3,6,7-naphthalenetetracarboxylic dianhydride, 1,4,5,8-naphthalenetetracarboxylic dianhydride, 2,2-bis{4-[4-(1,2-dicarboxy)phenoxy]phenyl}propane dianhydride, 2,2-bis{4-[3-(1,2-dicarboxy)phenoxy]phenyl}propane dianhydride, bis{4-[4-(1,2-dicarboxy)phenoxy]phenyl}ketone dianhydride, bis{4-[3-(1,2-dicarboxy)phenoxy]phenyl}ketone dianhydride, 4,4′-bis[4-(1,2-dicarboxy)phenoxy]biphenyl dianhydride, 4,4′-bis[3-(1,2-dicarboxy)phenoxy]biphenyl dianhydride, bis{4-[4-(1,2-dicarboxy)phenoxy]phenyl}ketone dianhydride, bis{4-[3-(1,2-dicarboxy)phenoxy]phenyl}ketone dianhydride, bis{4-[4-(1,2-dicarboxy)phenoxy]phenyl}sulfone dianhydride, bis{4-[3-(1,2-dicarboxy)phenoxy]phenyl}sulfone dianhydride, bis{4-[4-(1,2-dicarboxy)phenoxy]phenyl}sulfide dianhydride, bis{4-[3-(1,2-dicarboxy)phenoxy]phenyl}sulfide dianhydride and the like.

It is preferable that the tetracarboxylic acid component contains 3,3′,4,4′-biphenyltetracarboxylic dianhydride and/or 2,3,3′,4′-biphenyltetracarboxylic dianhydride which may be contained in an amount of 10 mol % or more, preferably 30 mol % or more, more preferably 50 mol % or more, further preferably 70 mol % or more and particularly preferably 80 mol % or more (may be contained in an amount of 100 mol %), in 100 mol % of the tetracarboxylic acid component. Further, the tetracarboxylic acid component may contain other aforementioned aromatic tetracarboxylic dianhydrides in the ranges in which the characteristics of the present invention are not impaired.

As the tetracarboxylic acid component, 3,3′,4,4′-biphenyltetracarboxylic dianhydride is used as a main component, whereby a polyimide excellent in breaking strength and elongation at break is obtained.

The polyimide of the present invention is obtained by reacting the diamine component and the tetracarboxylic acid component, as described above. As the production method, known methods may be adopted. For example, a polyimide may be prepared by a method comprising preparing a polyimide precursor by reacting the tetracarboxylic acid component with the diamine component in an organic solvent, and thereafter subjecting the precursor to chemical imidization or thermal imidization, or a method comprising direct imidization by reacting the tetracarboxylic acid component with the diamine component in an organic solvent or directly.

As the production method of the polyimide and polyimide precursor, all known methods may be used. The polyimide precursor may be usually prepared by reacting tetracarboxylic dianhydride with diamine in a substantially equimolar amount or in an excessive amount of any one of the tetracarboxylic dianhydride or the diamine component (any one of components is preferably 100 mol %, while the other component is preferably from 100 to 110 mol %, more preferably from 100 to 107 mol % and further preferably from 100 to 105 mol %) in an organic solvent, and stirring these materials until the polymerization reaction of the tetracarboxylic dianhydride and the diamine is (almost) completed under controlled temperature conditions. The polyimide precursor solution can be usually obtained at a concentration of 1 to 35 weight %, preferably 5 to 30 weight % and further 7 to 25 weight %. In this range of concentration, a suitable molecular weight and a suitable solution viscosity can be obtained.

Since A in the formula is a biphenylene group in the diamine compound of the formula (1), the solubility of the diamine component is extremely low and the diamine compound is hardly synthesized as compared to a conventional compound in which A is a phenylene group. However, as illustrated in Examples to be described below, unexpectedly, when the diamine compound of the formula (1) and the tetracarboxylic acid component are reacted, the polyimide precursor solution with suitable viscosity and with good stability in storage is easily obtained, and accordingly a film can be easily prepared.

As the polymerization method of the polyimide precursor, known methods may be used.

The diamine component and the tetracarboxylic dianhydride for providing a polyimide precursor are polymerized at a temperature of 0 to 100° C., preferably 5 to 50° C., in an organic solvent to give a polyimide precursor solution (so long as a uniform solution state is maintained, it may be partly imidized), and, if necessary, a plurality of polyimide precursor solutions are mixed, and the resulting mixed solution is coated into a coating film or formed into a film which is then dried, imidized and heat-dried (cured), whereby a polyimide can be prepared. The highest heating temperature for heat-drying is preferably in the range of 350 to 600° C., further preferably in the range of 400 to 550° C. and particularly preferably in the range of 400 to 500° C.

As a method for preparing a polyimide precursor, known methods can be used. Examples thereof include:

1) a method in which a carboxylic dianhydride component and a diamine component, each in an equimolar amount, may be reacted in an organic solvent, or may be reacted in an excessive amount of an acid or a diamine depending on the situation; and

2) a method in which a carboxylic dianhydride component and a diamine component represented by the general formula (1), each in an almost equimolar amount, are reacted in an organic solvent to prepare a polyimide precursor solution A; while a carboxylic dianhydride component and a diamine component other than the diamine represented by the general formula (1) are respectively reacted in an almost equimolar amount in an organic solvent to prepare a polyimide precursor solution B; and the polyimide precursor solutions A and B may be mixed and, if necessary, further polymerized. In this method, one of them may contain an excessive amount of an acid and the other may contain an excessive amount of diamine, depending on the situation.

When the amine terminal of the polyimide precursor needs to be capped, there may be added dicarboxylic anhydride, for example, phthalic anhydride and its substituted compound (for example, 3-methyl or 4-methylphthalic anhydride), hexahydrophthalic anhydride and its substituted compound, succinic anhydride and its substituted compound, or the like. For example, a small amount of phthalic anhydride may be added.

Furthermore, for the purpose of promoting imidization, an imidization agent may be added to the polyimide precursor solution. For example, there may be used imidazole, 1-methylimidazole, 2-methylimidazole, 1,2-dimethylimidazole, 2-phenylimidazole, benzimidazole, isoquinoline, substituted-pyridine and so on in a ratio of 0.05 to 10% by mass and particularly 0.1 to 2% by mass based on the polyimide precursor. Imidization can be completed at a relatively low temperature by using these compounds.

In the polyimide of the present invention, the carboxylic dianhydride component and a specific diamine component may have a block structure or a random structure.

To make a film from the polyimide of the present invention, for the purpose of suppressing gelation of the film, there may be added a phosphorus stabilizer, for example, triphenyl phosphite, triphenyl phosphate or the like in the range of 0.01 to 1% based on the solid content (polymer) concentration during polymerization of a polyamic acid.

The examples of the organic solvents used for the production of a polyimide precursor include N-methyl-2-pyrrolidone, N,N-dimethylformamide, N,N-dimethylacetamide, N,N-diethylacetamide, dimethyl sulfoxide, hexamethylphosphoramide, N-methylcaprolactam and the like. These organic solvents may be used alone or in combination of two or more.

The polyimide of the present invention allows the manufacturing of a film having characteristics as follows:

1) a water absorption percentage is 1.3% or less, and at the same time a hygroscopic expansion coefficient is preferably 10 ppm or less;

2) a water absorption percentage is 1.0% or less and preferably 0.9% or less, and a hygroscopic expansion coefficient is 7 ppm or less; or

3) a water absorption percentage is 0.7% or less, and at the same time a hygroscopic expansion coefficient is preferably 5 ppm or less.

Further, it allows the manufacturing of a film having elongation at break of 12% or more, preferably 14% or more and further preferably 15% or more, in addition to the aforementioned characteristics.

The polyimide of the present invention can be used as a coating agent or for a film (an uncured film is heat-treated using a pin tenter and substantially stretched).

When the polyimide of the present invention is used for a film, a thickness of the film is from about 3 to 200 μm, or when it is applied as a coating agent, its thickness is from about 0.1 to 2 μm.

Furthermore, the polyimide of the present invention can also be used for a modified polyimide layer for a surface layer on a core layer composed of a heat resistant polyimide. In this case, a polyimide precursor solution for providing a polyimide core layer composed of a heat resistant polyimide is cast on a support which is then dried to form a self-supporting film. On one side thereof, the polyimide precursor solution for providing the polyimide of the present invention is applied or sprayed such that a thickness after drying is from about 0.1 to 2 μm and dried, and, if necessary, on the other side, the polyimide precursor solution is applied or sprayed such that a thickness after drying is from about 0.1 to 2 μm and dried. The resulting material is heated to remove the solvent and imidized, and, if necessary, heat-dried (cured) at the highest heating temperature of 350 to 600° C., whereby a laminated polyimide film with at least one side subjected to modification can be prepared. This laminated polyimide film has a thickness of preferably from about 5 to 150 μm and particularly preferably from about 10 to 125 μm.,

The examples of the polyimide in the heat resistant polyimide layer of the laminated polyimide film may include:

i) a polyimide obtained by polymerizing and imidizing an aromatic tetracarboxylic dianhydride component composed of 3,3′,4,4′-biphenyltetracarboxylic dianhydride of 7.5 to 100 mol % and pyromellitic dianhydride of 0 to 92.5 mol %, and a diamine component composed of p-phenylenediamine of 15 to 100 mol % and 4,4′-diaminodiphenyl ether of 0 to 85 mol %, and, if necessary, heat-drying (curing) at the highest heating temperature of 350 to 600° C.;

ii) a polyimide obtained by polymerizing and imidizing an acid component of pyromellitic dianhydride and a diamine component of 4,4′-diaminodiphenyl ether and p-phenylenediamine in the ratio (molar ratio) of 4,4′-diaminodiphenyl ether to p-phenylenediamine of 90/10 to 10/90, and, if necessary, heat-drying (curing) at the highest heating temperature of 350 to 600° C.; and

iii) a polyimide obtained by polymerizing and imidizing aromatic tetracarboxylic dianhydride of 3,3′,4,4′-biphenyltetracarboxylic dianhydride of 7.5 to 100 mol % and pyromellitic dianhydride of 0 to 92.5 mol %, and a diamine component containing o-tolidine or m-tolidine, and, if necessary, heat-drying (curing) at the highest heating temperature of 350 to 600° C.

A laminate having a base material on at least one side of the polyimide of the present invention can be manufactured, by laminating at least one side of the polyimide of the present invention and a base material by pressing or pressure heating (lamination method) directly or via an adhesive to laminate them.

On at least one side of the polyimide of the present invention is formed a metal layer film using a thin film forming method and an electroplating method, whereby a laminate can be prepared.

Furthermore, the polyimide precursor solution for providing the polyimide of the present invention may be cast on a base material such as a metal foil or the like, and then imidization is completed chemically or by heat-drying, whereby a laminate can also be obtained.

The polyimide of the present invention may be formed into a laminated film. Thereafter, the polyimide layer of the present invention and a base material are pressed or pressure heated (lamination method) directly or via an adhesive to laminate them, whereby a laminate having a base material on at least one side thereof can be prepared.

A metal layer film may be formed, using a thin film forming method and an electroplating method, on the side of the polyimide layer of the present invention of the laminated polyimide film, whereby a laminate can be prepared.

In the lamination method, a heat resistant adhesive layer is formed on one side or both sides of the polyimide film of the present invention, and further a metal foil is laid (piled) thereon which is then heat pressurized, whereby a laminate can be obtained.

The heat resistant adhesive is not specifically limited so long as it is a heat resistant adhesive used in the field of electronics, and there may be exemplified a polyimide adhesive, an epoxy-modified polyimide adhesive, a phenolic resin-modified epoxy resin adhesive, an epoxy-modified acrylic resin adhesive, an epoxy-modified polyamide adhesive and the like. This heat resistant adhesive layer itself may be formed by any method which may be used in the field of electronics. For example, an adhesive solution may be applied to the aforementioned polyimide film or a formed product, and dried. Alternatively, they may be laminated with a film-form adhesive that is separately formed.

As the base material, there may be exemplified metal foil of single metal or alloy, such as copper, aluminum, gold, silver, nickel or stainless steel, metal plating layer (many known technologies can be suitably applied, such as a metal deposition base layer—metal plating layer, a chemical metal plating layer and the like) and the like. The preferred examples thereof include a rolled copper foil, an electrolytic copper foil, a copper plating layer and the like. A thickness of the metal foil is not specifically limited, and is preferably from 0.1 μm to 10 mm, further preferably from 1 to 50 μm and particularly preferably from 5 to 18 μm.

The laminate may further be bonded to other base materials, for example, ceramic, glass substrate, silicon wafer, or formed products of similar or different metals, a polyimide film or the like by the use of a heat resistant adhesive.

According to suitable examples of the present invention, since the water absorption percentage of the polyimide film is small, foaming or peeling hardly occurs at the adhesion interface even though a laminate using this film is treated at a high temperature such as a solder bath at 280° C. or the like.

The polyimide film of the present invention or the laminate having at least one layer of the polyimide of the present invention can be suitably used as a film for TAB, a board for an electronic component and a wiring board. It can be suitably used, for example, as a printed circuit board, a power circuit board, a flexible heater and a board for a resistor. Furthermore, it is useful for an insulating film, a protective film or the like to be formed on a material having a low linear expansion coefficient, such as a base material for LSI or the like.

The novel diamine compound of the present invention will be described hereinafter.

The diamine compound of the present invention is a compound represented by the following formula (1):

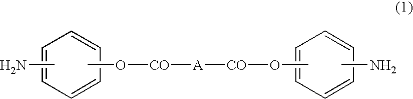

In the formula (1), A is a biphenylene group which may have a substituent and preferably a 4,4′-biphenylene group represented by the formula (A1):

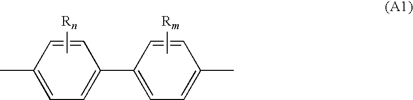

wherein, n and m each represents the number of substituents Rs on each ring and each independently represents 0, 1, 2, 3 or 4, and when both n and m are 0, the compound of the formula (A1) represents an unsubstituted 4,4′-biphenylene group. R represents an alkyl group having up to 4 carbon atoms, and is preferably methyl group, ethyl group, propyl group or the like. In case R occurs more than once in the formula (A1), each R, independently one another, has the same meaning as defined above. A is preferably a biphenylene group represented by the formula (A2), (A3), (A4) or (A5), and most preferably a group represented by the formula (A2),

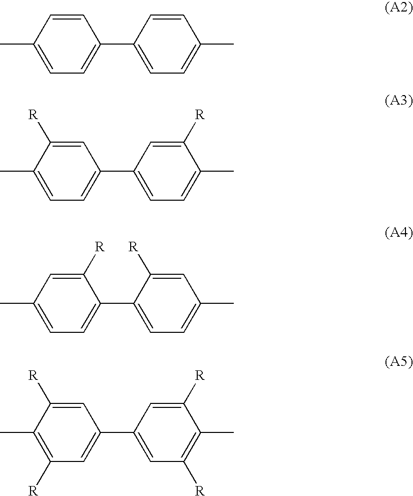

wherein, R has the same meaning as defined above.

A terminal —NH₂ group of the compound of the formula (1) is bonded to the phenylene group at the ortho position, the meta position or the para position relative to an —O— group. Preferably, the terminal —NH₂ group of the compound of the formula (1) is preferably bonded to the phenylene group at the para position relative to the —O— group.

The diamine compound represented by the formula (1) of the present invention is preferably biphenyl-4,4′-dicarboxylic acid bis(4-aminophenyl)ester represented by the following formula (1a).

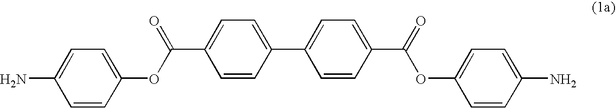

The compound is useful as a raw material of the polyimide as described above. In addition, the compound can also be used as a raw material of the polyamide or the like. The compound is novel, and its existence and production method have not been known at all.

The production method of the compound of the formula (1) will be described separately, based on the difference of reaction process, in Production Method I and in Production Method II.

First Production Method (Production Method I)

The compound of the formula (1) can be synthesized as illustrated below. That is, the diamine compound of the formula (1) can be obtained by reacting the biphenyldicarbonyl halide derivative represented by the general formula (2):

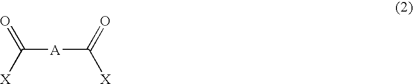

(wherein, A is the same as described above; and X represents a halogen atom.)

with nitrophenol in the presence of a base to give biphenyl-dicarboxylic acid bis(nitrophenyl)ester represented by the general formula (3):

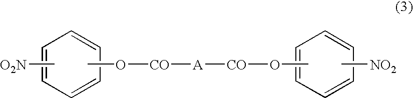

, and subsequently reducing biphenyl-dicarboxylic acid bis(nitrophenyl)ester represented by the general formula (3).

The reaction of the present invention is further described in detail with reference to the synthesis of biphenyl-4,4′-dicarboxylic acid bis(4-aminophenyl)ester. However, compounds in which the group A represents other groups can also be synthesized in the same manner. The reaction of the present invention comprises two reaction steps, i.e., (A) an esterification reaction and (B) a reduction reaction, as shown in Reaction Step Formula (1).

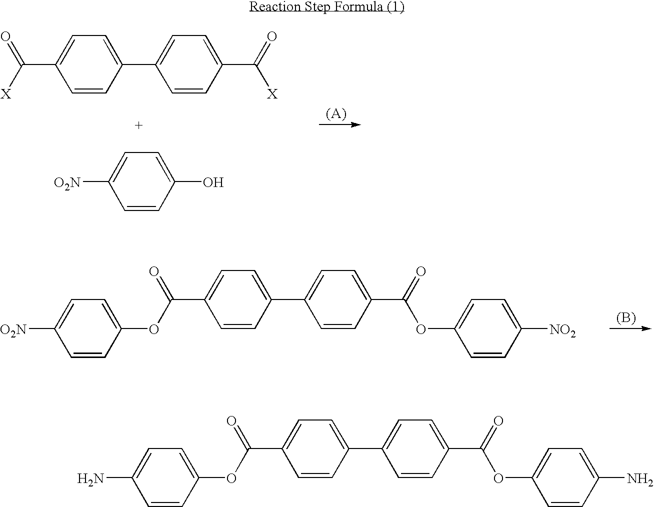

(wherein, X has the same meaning as defined above.)

These two reactions will be illustrated in order.

(A) Esterification Reaction

The esterification reaction refers to a reaction to obtain biphenyl-4,4′-dicarboxylic acid bis(4-nitrophenyl)ester by reacting biphenyldicarbonyl halide with 4-nitrophenol. The biphenyldicarbonyl halide to be used is represented by the above general formula (2), and preferably biphenyl-4,4′-dicarbonyl halide. The examples of halogen atom represented by X include fluorine atom, chlorine atom, bromine atom and iodine atom. Preferably used are chlorine atom and a bromine atom.

As the base used in the esterification reaction, there may be exemplified alkali hydrides such as sodium hydride, potassium hydride, lithium hydride and the like; alkali metal hydroxides or alkaline earth metal hydroxides such as sodium hydroxide, potassium hydroxide, calcium hydroxide and the like; alkali metal carbonates or alkaline earth metal carbonates such as lithium carbonate, sodium carbonate, potassium carbonate, rubidium carbonate, caesium carbonate, calcium carbonate and the like; alkali metal hydrogen carbonates or alkaline earth metal hydrogen carbonates such as sodium hydrogen carbonate, potassium hydrogen carbonate, calcium hydrogen carbonate and the like; amines such as triethylamine, diisopropylamine, n-butylamine, N-methylpiperidine, N-methylmorpholine and the like; and pyridines such as pyridine, dimethylpyridine and the like. Preferably used are alkali metal hydrides, alkali metal carbonates, amines and pyridines, and further preferably used are sodium hydride, sodium carbonate, triethylamine and pyridine. These bases may be used alone or in combination of two or more.

The amount of the base used is preferably from 1 to 10 mol and further preferably from 1.5 to 5.0 mol based on 1 mol of the biphenyldicarbonyl halide.

The amount of 4-nitrophenol used in the esterification reaction is preferably from 1 to 10 mol and further preferably from 1.5 to 5.0 mol based on 1 mol of the biphenyldicarbonyl halide.

The esterification reaction is preferably carried out in the presence of an organic solvent. As the solvent to be used, it is not specifically limited so long as it does not hinder the reaction, and there may be exemplified ethers such as diethyl ether, diisopropyl ether, dioxane, ethylene glycol dimethyl ether, ethylene glycol diethyl ether, tetrahydrofuran and the like; amides such as N,N-dimethylformamide, N,N-dimethylacetamide, N-methyl-2-pyrrolidone and the like; ureas such as N,N′-dimethyl-2-imidazolidinone and the like; nitriles such as acetonitrile, propionitrile, benzonitrile and the like; ketones such as acetone, methylethyl ketone, methylisobutyl ketone and the like; aromatic hydrocarbons such as benzene, toluene, xylene, cumene and the like; and halogenated aliphatic hydrocarbons such as methylene chloride, dichloroethane and the like. Preferably used are ethers, amides, nitrites and ketones, and further preferably used are ethers and amides. These solvents may be used alone or in combination of two or more.

The amount of the aforementioned solvent to be used is properly adjusted depending on a degree of uniformity of the reaction solution or condition of stirring, and is preferably from 1 to 100 ml and further preferably from 10 to 80 ml based on 1 g of the biphenyldicarbonyl halide.

The esterification reaction is carried out by the method in which, for example, the biphenyldicarbonyl halide, 4-nitrophenol and a base are mixed, and these materials may be stirred. A reaction temperature during the reaction is preferably from 0 to 200° C. and further preferably from 10 to 100° C., while a reaction pressure is not specifically limited.

The resulting biphenyl-4,4′-dicarboxylic acid bis(4-nitrophenyl)ester is isolated and purified by, for example, a general method such as extraction, filtration, concentration, recrystallization, column chromatography or the like, after completion of the reaction. Alternatively, the produced biphenyl-4,4′-dicarboxylic acid bis(4-nitrophenyl)ester may be used for the following reduction reaction without isolating and purifying it.

(B) Reduction Reaction

The reduction reaction refers to a reaction to obtain biphenyl-4,4′-dicarboxylic acid bis(4-aminophenyl)ester by reducing biphenyl-4,4′-dicarboxylic acid bis(4-nitrophenyl)ester. This reduction reaction is not specifically limited so long as it is a method comprising converting a nitro group into an amino group, and a preferred method comprises reaction with hydrogen in the presence of a metal catalyst. The metal atom herein used may include, for example, nickel, palladium, platinum, rhodium, ruthenium, cobalt, copper and the like. The metal atom may be used as it is or in the state of a metal oxide. Furthermore, the metal atom as it is or a metal oxide may also be used in supported forms on carriers, such as carbon, barium sulfate, silica gel, alumina, celite or the like, while nickel, cobalt, copper or the like may also be used as a Raney catalyst.

The amount of the aforementioned catalyst to be used is preferably from 0.01 to 10% by mass and further preferably from 0.05 to 5% by mass in terms of the metal atom based on biphenyl-4,4′-dicarboxylic acid bis(4-nitrophenyl)ester. Herein, these metal catalysts may be used alone or in combination of two or more, and they may be dry products or wet products.

The amount of hydrogen used in this reaction is preferably from 1 to 20 mol and further preferably from 4 to 10 mol based on 1 mol of the biphenyl-4,4′-dicarboxylic acid bis(4-nitrophenyl)ester. Herein, hydrogen gas may be diluted with a gas which is inert to the reaction, such as nitrogen, argon or the like.

The reduction reaction is preferably carried out in the presence of a solvent. As the solvent to be used, it is not specifically limited so long as it does not hinder the reaction, and there may be exemplified water; alcohols such as methanol, ethanol, n-propyl alcohol, isopropyl alcohol, n-butyl alcohol, isobutyl alcohol, t-butyl alcohol and the like; amides such as N,N-dimethylformamide, N,N-dimethylacetamide, N-methyl-2-pyrrolidone and the like; ureas such as N,N′-dimethyl-2-imidazolidinone and the like; and ethers such as diethyl ether, diisopropyl ether, tetrahydrofuran and the like. Preferably used are alcohols and amides, and further preferably used are N,N-dimethylformamide, N,N-dimethylacetamide and N-methyl-2-pyrrolidone. These solvents may be used alone or in combination of two or more.

The amount of the aforementioned solvent to be used is properly adjusted depending on a degree of uniformity of the reaction solution or condition of stirring, and is preferably from 1 to 100 ml and further preferably from 5 to 50 ml based on 1 g of the biphenyl-4,4′-dicarboxylic acid bis(4-nitrophenyl)ester.

The reduction reaction is carried out by the method in which, for example, biphenyl-4,4′-dicarboxylic acid bis(4-nitrophenyl)ester and a solvent are mixed in the presence of a metal catalyst, and these materials are reacted with hydrogen while stirring. A reaction temperature during the reaction is preferably from 0 to 200° C. and further preferably from 10 to 100° C., while a reaction pressure is preferably from 0.1 to 20 MPa and further preferably from 0.1 to 5 MPa. The reaction may be carried out by allowing hydrogen to flow or in a sealed reaction vessel. In case hydrogen is allowed to flow, its flow rate is properly adjusted depending on the capacity of the reaction mixture or the size of the reaction vessel.

The resulting biphenyl-4,4′-dicarboxylic acid bis(4-aminophenyl)ester is isolated and purified by, for example, a general method such as extraction, filtration, concentration, recrystallization, column chromatography or the like, after completion of the reaction.

Second Production Method (Production Method II)

The Second Production Method of the diamine compound represented by the formula (1) is illustrated. In this Production Method, the aforementioned general formula (1):

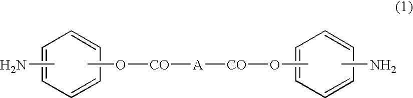

is obtained by reacting a biphenyl carbonyl derivative represented by the general formula (21):

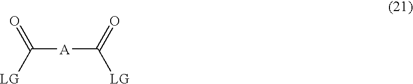

(wherein, A is the same as described above; and LG is a leaving group.) with aminophenol in the presence of a base.

Aminophenol is preferably 4-aminophenol. Further, in the formula (21), a leaving group LG may be a group which can be exchanged with aminophenoxy:

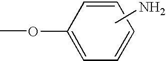

in aminophenol. LG is preferably a phenoxy group which may have a substituent and a group represented by the following formula (31):

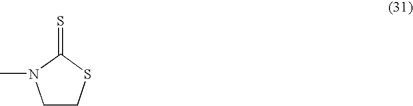

Hereinafter, these cases will be described in detail.

Production Method II-1

(LG is a phenoxy group which may have a substituent)

In the compound of the formula (21), when LG is a phenoxy group which may optionally have a substituent, the general formula (21) is represented by the general formula (22):

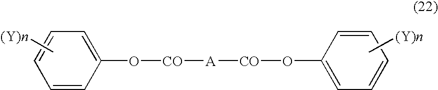

Production Method II 1 refers to a method in which the diamine compound represented by the general formula (1) is obtained by reacting a biphenyl-dicarboxylic acid bis(aryl)ester compound represented by the general formula (22) with aminophenol in the presence of a base.

In the formula (22), A has the same meaning as defined above, preferably the aforementioned formulae (A2) to (A5), and most preferably an unsubstituted 4,4′-biphenylene group of the formula (A2). Y preferably represents a halogen atom, a nitro group, a trifluoromethyl group, a cyano group or an acetyl group, and further preferably a halogen atom or a nitro group. n preferably represents an integer of 0 to 3.

The compound represented by the formula (22) is a novel compound except for biphenyl-4,4′-dicarboxylic acid diphenyl ester, biphenyl-4,4′-dicarboxylic acid bis(2-chlorophenyl)ester and biphenyl-4,4′-dicarboxylic acid bis(2-nitrophenyl)ester

The biphenyl-dicarboxylic acid bis(aryl)ester compound of the formula (22) is obtained by reacting the biphenyldicarbonyl halide derivative represented by the aforementioned general formula (2):

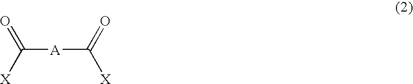

(wherein, A is the same as described above; and X represents a halogen atom.)
   a hydroxyaryl compound represented by the general formula (23):

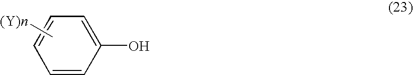

(wherein, Y and n represent the same meaning as those defined in the formula (22))
   and a base,.

Hereinafter, this reaction will be further described in detail with reference to the synthesis of biphenyl-4,4′-dicarboxylic acid bis(4-aminophenyl)ester. However, compounds in which the group A represents other groups can also be synthesized in the same manner.

The entire reaction from the synthesis of the raw material comprises two reactions, i.e., (A) a reaction for obtaining a biphenyl-4,4′-dicarboxylic acid bis(aryl)ester compound by reacting biphenyl-4,4′-dicarbonyl halide with a hydroxyaryl compound (hereinafter referred to as an esterification reaction), and (B) a reaction for obtaining biphenyl-4,4′-dicarboxylic acid bis(4-aminophenyl)ester by reacting the biphenyl-4,4′-dicarboxylic acid bis(aryl)ester compound with 4-aminophenol (hereinafter referred to as an ester exchange reaction) as shown in Reaction Step Formula (2),

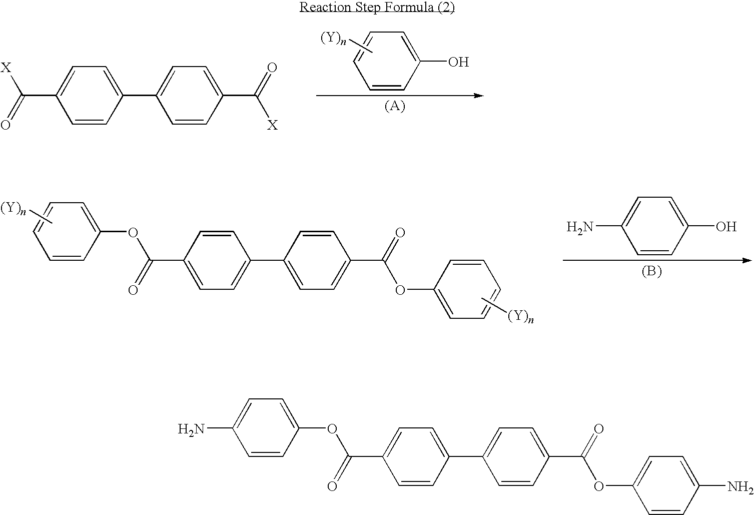

wherein, in the formula, X, Y and n have the same meaning as defined above.

Hereinafter, these two reactions will be described in order.

(A) Esterification Reaction

In the esterification reaction, the biphenyl-4,4′-dicarboxylic acid bis(aryl)ester compound is a compound obtained by reacting biphenyldicarboxylic acid halide represented by the aforementioned general formula (2), the hydroxyaryl compound and a base (see Reference Examples C-1 to C-3 to be described below). In the general formula (2), X is a halogen atom, for example, a fluorine atom, a chlorine atom, a bromine atom or an iodine atom, and preferably a chlorine atom or a bromine atom.

The hydroxyaryl compound to be used in this reaction is represented by the aforementioned general formula (23). In the general formula (23), Y is preferably, for example, a fluorine atom, a chlorine atom, a bromine atom, an iodine atom or a nitro group, and further preferably a chlorine atom, a bromine atom or a nitro group. n represents the number of substituents, and is specifically 0 to 3 and preferably 0 to 2.

The aforementioned hydroxyaryl compound is specifically phenol or phenols substituted with 1 to 3 halogen atoms or nitro groups. A substitution position of the substituted phenols with 1 to 3 group Ys is desirably at least one substitution position selected from the group consisting of the 2 position, the 4 position and the 6 position.

The amount of the aforementioned hydroxyaryl compound is preferably from 2.0 to 20 mol and more preferably from 2.0 to 10 mol based on 1 mol of the biphenyl-4,4′-dicarbonyl dihalide.

As the base to be used in the esterification reaction, there may be exemplified alkali hydrides such as sodium hydride, potassium hydride, lithium hydride and the like; alkali metal hydroxides or alkaline earth metal hydroxides such as sodium hydroxide, potassium hydroxide, calcium hydroxide and the like; alkali metal carbonates or alkaline earth metal carbonates such as lithium carbonate, sodium carbonate, potassium carbonate, rubidium carbonate, caesium carbonate, calcium carbonate and the like; alkali metal hydrogen carbonates or alkaline earth metal hydrogen carbonates such as sodium hydrogen carbonate, potassium hydrogen carbonate, calcium hydrogen carbonate and the like; amines such as triethylamine, diisopropylamine, n-butylamine, N-methylpiperidine, N-methylmorpholine and the like; and pyridines such as pyridine, dimethylpyridine and the like. Preferably used are alkali metal hydrides, alkali metal carbonates, amines and pyridines, and further preferably used are sodium hydride, sodium carbonate, triethylamine and pyridine. These bases may be used alone or in combination of two or more.

The amount of the aforementioned base to be used is preferably from 1 to 20 mol and more preferably from 2 to 10 mol based on 1 mol of the biphenyl-4,4′-dicarbonyl halide.

The esterification reaction is preferably carried out in the presence of an organic solvent. As the solvent to be used, it is not specifically limited so long as it does not hinder the reaction, and there may be exemplified ethers such as diethyl ether, diisopropyl ether, dioxane, ethylene glycol dimethyl ether, ethylene glycol diethyl ether, tetrahydrofuran and the like; amides such as N,N-dimethylformamide, N,N-dimethylacetamide, N-methyl-2-pyrrolidone and the like; ureas such as N,N′-dimethyl-2-imidazolidinone and the like; nitrites such as acetonitrile, propionitrile, benzonitrile and the like; ketones such as acetone, methylethyl ketone, methylisobutyl ketone and the like; aromatic hydrocarbons such as benzene, toluene, xylene, cumene and the like; and halogenated aliphatic hydrocarbons such as methylene chloride, dichloroethane and the like. Preferably used are ethers, amides, nitriles and ketones, and further preferably used are ethers and amides. These solvents may be used alone or in combination of two or more.

The amount of the aforementioned solvent to be used is properly adjusted depending on a degree of uniformity of the reaction solution or condition of stirring, and is preferably from 1 to 100 ml and more preferably from 2 to 50 ml based on 1 g of the biphenyl-4,4′-dicarbonyl halide.

The esterification reaction is carried out by the method in which, for example, the biphenyl-4,4′-dicarbonyl halide, the hydroxyaryl compound and a solvent are mixed in the presence of a base, and these materials may be stirred. A reaction temperature during the reaction is preferably from −20 to 250° C., more preferably from 0 to 150° C. and particularly preferably from 15 to 120° C., while a reaction pressure is not specifically limited.

The resulting biphenyl-4,4′-dicarboxylic acid bis(aryl)ester compound is isolated and purified by, for example, a general method such as extraction, filtration, concentration, recrystallization, column chromatography or the like, after completion of the reaction. Alternatively, the produced biphenyl-4,4′-dicarboxylic acid bis(aryl)ester compound may be used for the following ester exchange reaction without particularly isolating and purifying it.

(B) Ester Exchange Reaction

The ester exchange reaction is carried out by reacting the biphenyl-4,4′-dicarboxylic acid bis(aryl)ester compound represented by the aforementioned general formula (22), more specifically the formula (22a):

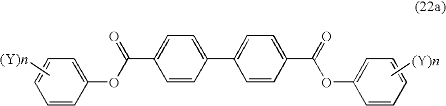

with aminophenol (preferably 4-aminophenol) in the presence of a base, to obtain a compound represented by the aforementioned general formula (1), more specifically the formula (la).

The amount of aminophenol to be used in this ester exchange reaction is preferably from 2.0 to 20 mol and more preferably from 2.0 to 10 mol based on 1 mol of the biphenyl-4,4′-dicarboxylic acid bis(aryl)ester compound.

As the base to be used in the ester exchange reaction, there may be exemplified triethylamine, 1,4-diazabicyclo[2,2,2]octane, pyridine, 1-methyl-1,3,4,6,7,8-hexahydro-2H-pyrimido[1,2-a]pyrimidine, N′-cyclohexyl-N,N,N,N-tetramethylguanidine or organic amine having a guanidine skeleton as a partial structure; organic amine having an amidine skeleton as a partial structure such as 1,8-diazabicyclo[5,4,0]-7-undecene, 1,5-diazabicyclo[4,3,1]-5-nonene and the like; inorganic carbonates such as lithium carbonate, sodium carbonate, potassium carbonate, rubidium carbonate, caesium carbonate and the like; inorganic hydrogen carbonates such as sodium hydrogen carbonate, potassium hydrogen carbonate and the like; alkali metal hydrides such as sodium hydride, potassium hydride, lithium hydride and the like; alkali metal hydroxides such as potassium hydroxide, sodium hydroxide and the like; and alkali metal alkoxides such as lithium methoxide, sodium methoxide, sodium t-butoxide, sodium ethoxide, potassium t-butoxide and the like (these may be used as a corresponding alcohol solution). Preferably used are alkali metal hydrides, alkali metal alkoxides and organic amine, and further preferably used are sodium hydride, sodium t-butoxide, potassium t-butoxide, and organic amine having an amidine skeleton as a partial structure such as 1,8-diazabicyclo[5,4,0]-7-undecene, 1,5-diazabicyclo[4,3,0]-5-nonene and the like. These bases may be used alone or in combination of two or more.

The amount of the base to be used is preferably from 0.005 to 2.5 mol, further preferably from 0.01 to 1.99 mol and particularly preferably from 0.1 to 1.0 mol based on 1 mol of the biphenyl-4,4′-dicarboxylic acid di(aryl)ester.

The ester exchange reaction is preferably carried out in the presence of a solvent. As the solvent to be used, it is not specifically limited so long as it does not hinder the reaction, and there may be exemplified ethers such as diethyl ether, diisopropyl ether, dioxane, ethylene glycol dimethyl ether, ethylene glycol diethyl ether, tetrahydrofuran and the like; ketones such as acetone, methylethyl ketone, methylisobutyl ketone and the like; aromatic hydrocarbons such as benzene, toluene, xylene, cumene and the like; halogenated aromatic hydrocarbons such as chlorobenzene, 1,2-dichlorobenzene, 1,3-dichlorobenzene, 1,4-dichlorobenzene and the like; amides such as N,N-dimethylformamide, N,N-dimethylacetamide, N-methyl-2-pyrrolidone and the like; ureas such as N,N′-dimethyl-2-imidazolidinone and the like; nitrites such as acetonitrile, propionitrile, benzonitrile and the like; nitro-aromatic hydrocarbons nitrobenzene and the like; and sulfoxides such as dimethyl sulfoxide and the like. Preferably used are ethers, halogenated aromatic hydrocarbons, ureas, nitrated aromatic hydrocarbons and sulfoxides. These solvents may be used alone or in combination of two or more.

The amount of the aforementioned solvent to be used is properly adjusted depending on a degree of uniformity of the reaction solution or condition of stirring, and is preferably from 1 to 100 ml and more preferably from 2 to 50 ml based on 1 g of the biphenyl-4,4′-dicarboxylic acid bis(aryl)ester compound.

The ester exchange reaction is carried out by the method in which, for example, the biphenyl-4,4′-dicarboxylic acid bis(aryl)ester compound and a solvent are mixed in the presence of a base, and these materials are stirred. A reaction temperature during the reaction is preferably from 50 to 250° C. and more preferably from 80 to 200° C., while a reaction pressure is not specifically limited.

However, in the aforementioned formula (22), when n is 0, namely, both ends are unsubstituted phenyl groups, preferably used is, for example, a method comprising stirring while removing the generated phenol from the reaction solution in the ester exchange reaction. A reaction temperature at that time is preferably from 50 to 250° C. and further preferably from 80 to 200° C., while a reaction pressure is not specifically limited, and is preferably from 0.6 to 70 kPa and further preferably from 1 to 40 kPa. A preferable aspect in this case include, for example, a method in which biphenyl-4,4′-dicarboxylic acid diphenyl ester, 4-aminophenol and a solvent are mixed in the presence of a base, and these materials are reacted while removing the generated phenol from the reaction solution at a reaction temperature of 50° C. to 250° C. and a reaction pressure of 0.6 to 70 kPa.

The resulting biphenyl-4,4′-dicarboxylic acid bis(4-aminophenyl)ester is isolated and purified by, for example, a general method such as extraction, filtration, concentration, recrystallization, crystallization, column chromatography or the like, after completion of the reaction.

Production Method I-2

(LG is a group represented by the formula (31))

In the compound of the formula (21), when LG is a group represented by the formula (31), the general formula (21) is represented by the general formula (32):

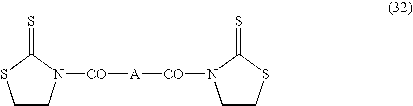

Production Method II-2 refers to a method in which the diamine compound represented by the general formula (1) is obtained by reacting a biphenyl carbamide compound represented by the general formula (32) with aminophenol in the presence of a base.

In the formula (32), A has the same meaning as defined above, preferably the aforementioned formulae (A2) to (A5), and most preferably an unsubstituted 4,4′-biphenylene group of the formula (A2).

The biphenyl carbamide compound of the formula (32) is a novel compound, and is obtained by reacting the biphenyldicarbonyl halide derivative represented by the aforementioned general formula (2):

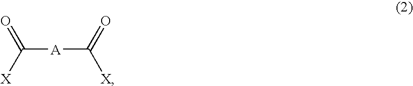

2-thiazoline-2-thiol {formula (33):

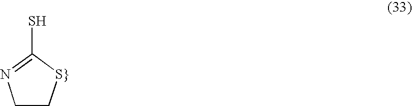

and a base.

Hereinafter, this reaction will be described in detail with reference to the synthesis of biphenyl-4,4′-dicarboxylic acid bis(4-aminophenyl)ester. However, compounds in which the group A represents other groups can also be synthesized in the same manner.

The entire reaction from the synthesis of the raw material comprises two reactions, i.e., (A) a reaction for obtaining a biphenyl carbamide compound by reacting biphenyldicarbonyl halide with 2-thiazoline-2-thiol (hereinafter referred to as an amidation reaction) and (B) a reaction for obtaining biphenyl-4,4′-dicarboxylic acid bis(4-aminophenyl)ester by reacting the biphenyl carbamide compound with 4-aminophenol (hereinafter referred to as an esterification reaction) as shown in Reaction Step Formula (3).

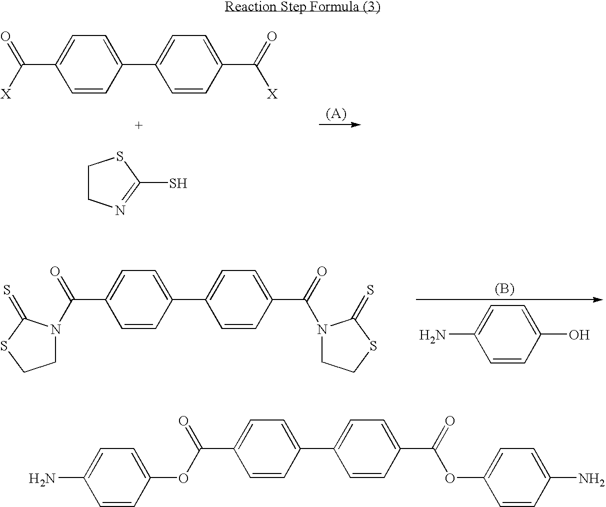

(wherein, X has the same meaning as defined above.)

Hereinafter, these two reactions will be described in order

(A) Amidation Reaction

The biphenyldicarbonyl halide to be used in this amidation reaction is represented by the aforementioned general formula (1). In the general formula (1), X has the same meaning as defined above, and as the halogen atom, there may be exemplified a fluorine atom, a chlorine atom, a bromine atom and an iodine atom, and preferably used are a chlorine atom and a bromine atom.

The amount of 2-thiazoline-2-thiol used in the amidation reaction is preferably from 1.6 to 20 mol and further preferably from 2.0 to 10 mol based on 1 mol of the biphenyldicarbonyl halide.

As the base to be used in the amidation reaction, there may be mentioned, for example, organic bases such as triethylamine, pyridine, 1,8-diazabicyclo[5,4,0]-7-undecene, 1,5-diazabicyclo[4,3,0]-5-nonene, 1,4-diazabicyclo[2,2,2]octane and the like; and inorganic bases such as lithium carbonate, sodium carbonate, potassium carbonate, rubidium carbonate, caesium carbonate, sodium hydrogen carbonate, potassium hydrogen carbonate and the like. Preferably used are organic bases. These bases may be used alone or in combination of two or more.

The amount of the aforementioned base to be used is preferably from 1 to 20 mol and further preferably from 2 to 10 mol based on 1 mol of the biphenyldicarbonyl halide.

The amidation reaction is carried out in the presence or absence of a solvent. As the solvent to be used, it is not specifically limited so long as it does not hinder the reaction, and there may be exemplified aromatic hydrocarbons such as benzene, toluene, xylene, cumene and the like; halogenated aliphatic hydrocarbons such as methylene chloride, 1,2-dichloroethane, 1,1-dichloroethane and the like; halogenated aromatic hydrocarbons such as chlorobenzene, 1,2-dichlorobenzene, 1,3-dichlorobenzene, 1,4-dichlorobenzene and the like; ethers such as diethyl ether, diisopropyl ether, dioxane, ethylene glycol dimethyl ether, ethylene glycol diethyl ether, tetrahydrofuran and the like; ketones such as acetone, methylethyl ketone, methylisobutyl ketone, cyclohexanone and the like; amides such as N,N-dimethylformamide, N,N-dimethylacetamide, N-methyl-2-pyrrolidone and the like; ureas such as N,N′-dimethyl-2-imidazolidinone and the like; nitrites such as acetonitrile, propionitrile, benzonitrile and the like, nitrobenzene, dimethyl sulfoxide and the like. Preferably used are ethers, amides, ureas, dimethyl sulfoxide and the like. These solvents may be used alone or in combination of two or more.

The amount of the aforementioned solvent to be used is properly adjusted depending on a degree of uniformity of the reaction solution or condition of stirring, and is preferably from 1 to 100 ml and further preferably from 2 to 50 ml based on 1 g of the biphenyldicarbonyl halide.

This amidation reaction is carried out by the method in which, for example, biphenyldicarbonyl halide, 2-thiazoline-2-thiol, a base and a solvent are mixed, and these materials may be stirred. A reaction temperature during the reaction is preferably from 0 to 150° C. and further preferably from 10 to 100° C., while a reaction pressure is not specifically limited.

The resulting biphenyl carbamide compound is isolated and purified by, for example, a general method such as extraction, filtration, concentration, recrystallization, column chromatography or the like, after completion of the reaction. Alternatively, the produced biphenyl carbamide compound may be used for the following esterification reaction without isolating and purifying it.

(B) Esterification Reaction

The aminophenol (preferably 4-aminophenol) used in this esterification reaction is preferably from 1.0 to 20 mol and further preferably from 2.0 to 10 mol based on 1 mol of the biphenyl carbamide compound.

As the base used in the esterification reaction, there may be exemplified organic bases such as 1,8-diazabicyclo[5,4,0]-7-undecene, 1,5-diazabicyclo[4,3,1]-5-nonene, 1,4-diazabicyclo[2,2,2]octane and the like; inorganic bases such as lithium carbonate, sodium carbonate, potassium carbonate, rubidium carbonate, caesium carbonate, sodium hydride, lithium hydride and the like; and metal alkoxides such as sodium methoxide, sodium ethoxide, potassium t-butoxide, sodium t-butoxide and the like. Preferably used are organic bases, metal alkoxides and sodium hydride, and further preferably used are 1,8-diazabicyclo[5,4,0]-7-undecene, potassium t-butoxide, sodium t-butoxide and sodium hydride. These bases may be used alone or in combination of two or more.

The amount of the aforementioned base to be used is preferably from 0.01 to 10 mol and further preferably from 0.1 to 5 mol based on 1 mol of the biphenyl carbamide compound.

This esterification reaction is carried out in the presence or absence of a solvent. As the solvent to be used, it is not specifically limited so long as it does not hinder the reaction, and there may be exemplified aromatic hydrocarbons such as benzene, toluene, xylene, cumene and the like; halogenated aliphatic hydrocarbons such as methylene chloride, 1,2-dichloroethane, 1,1-dichloroethane and the like; halogenated aromatic hydrocarbons such as chlorobenzene, 1,2-dichlorobenzene, 1,3-dichlorobenzene, 1,4-dichlorobenzene and the like; ethers such as diethyl ether, diisopropyl ether, dioxane, ethylene glycol dimethyl ether, ethylene glycol diethyl ether, tetrahydrofuran and the like; amides such as N,N-dimethylformamide, N,N-dimethylacetamide, N-methyl-2-pyrrolidone and the like; ureas such as N,N′-dimethyl-2-imidazolidinone and the like; nitrites such as acetonitrile, propionitrile, benzonitrile and the like, nitrobenzene, dimethyl sulfoxide and the like. Preferably used are ethers, amides, ureas and dimethyl sulfoxide. These solvents may be used alone or in combination of two or more.

The amount of the aforementioned solvent to be used is properly adjusted depending on a degree of uniformity of the reaction solution or condition of stirring, and is preferably from 1 to 100 ml and further preferably from 2 to 50 ml based on 1 g of the biphenyl carbamide compound.

The esterification reaction is carried out by the method in which, for example, the biphenyl carbamide compound, 4-aminophenol, a base and a solvent are mixed, and these materials may be stirred. A reaction temperature during the reaction is preferably from −50 to 100° C. and further preferably from −20 to 60° C., while a reaction pressure is not specifically limited.

The resulting biphenyl-4,4′-dicarboxylic acid bis(4-aminophenyl)ester is isolated and purified by, for example, a general method such as extraction, filtration, concentration, recrystallization, column chromatography or the like, after completion of the reaction

### Examples

The present invention will be more specifically described with reference to Examples and Comparative Examples below.

### Synthesis Examples of Diamine Compounds by Production Method I

### Example A-1

### (A) Synthesis of biphenyl-4,4′-dicarboxylic acid bis(4-nitrophenyl)ester

To a flask having an inner volume of 2,000 ml and equipped with a stirring device, a thermometer and a dropping funnel were added 41.87 g (0.15 mol) of 4,4′-biphenyldicarbonyl chloride, 45.91 g (0.33 mol) of 4-nitrophenol and 1,050 ml of tetrahydrofuran. A solution of 37.95 g (0.38 mol) of triethylamine dissolved in 75 ml of tetrahydrofuran was added to the mixed solution at 25° C. over a period of 2 hours, and then the resulting mixture was reacted at 45° C. for 3 hours. After completion of the reaction, the reaction solution was cooled to 25° C. and then filtered. The filtered residue was dried to obtain 112 g of yellow powder. The obtained powder and 1,750 ml of N,N-dimethylformamide were added to a flask having an inner volume of 2,000 ml and equipped with a stirring device and a thermometer, and the mixture was stirred at 95° C. for 20 minutes. After completion of stirring, the mixed solution was cooled to 25° C. and then filtered. The filtered residue was washed with 1,200 ml of water and 600 ml of tetrahydrofuran in order The filtered residue was dried to obtain 57.4 g (0.12 mol) of biphenyl-4,4′-dicarboxylic acid bis(4-nitrophenyl)ester (Isolation yield; 80% on the basis of 4,4′-biphenyldicarbonyl chloride) as colorless powder.

Physical properties of biphenyl-4,4′-dicarboxylic acid bis(4-nitrophenyl)ester were as follows.

¹H-NMR(DMSO-d₆,δ(ppm)); 7.68(4H,d,J=9.3 Hz), 8.08(4H,d,J=8.6 Hz), 8.31(4H,d,J=8.6 Hz), 8.39(4H,d,J=9.3 Hz)

### Example A-2

### (B) Synthesis of biphenyl-4,4′-dicarboxylic acid bis(4-aminophenyl)ester (hereinafter abbreviated as APBP)

To a flask having an inner volume of 500 ml and equipped with a stirring device, a thermometer and a reflux condenser were added 30.0 g (61.9 mmol) of biphenyl-4,4′-dicarboxylic acid bis(4-nitrophenyl)ester prepared in the same manner as in Example A-1, 330 ml of N,N-dimethylformamide and 6.29 g (150 mg calculated as palladium atom) of 5% by mass palladium/carbon (AD catalyst manufactured by Kawaken Fine Chemicals Co., Ltd., 52.33% by mass wet product). The flask was equipped with a balloon charged with hydrogen gas and reaction was carried out at 80° C. for 7 hours with stirring. After completion of the reaction, the reaction solution was cooled to 60° C. and then filtered. 300 ml of water was added to the filtrate, and the mixture was stirred at 25° C. for 0.5 hour. The precipitated crystal was filtered and the filtered residue was washed with 50 ml of N,N-dimethylformamide, 100 ml of water and 100 ml of methanol in order, and then dried to obtain 24.3 g (57.3 mmol) of biphenyl-4,4′-dicarboxylic acid bis(4-aminophenyl)ester with a purity of 97.0% (areal percentage by high performance liquid chromatography) as skin color powder (Reaction yield; 93% on the basis of biphenyl-4,4′-dicarboxylic acid bis(4-nitrophenyl)ester).

18.9 g of the obtained biphenyl-4,4′-dicarboxylic acid bis(4-aminophenyl)ester and 90 ml of dimethyl sulfoxide were mixed and recrystallized to obtain 12.6 g (29.7 mmol) of biphenyl-4,4′-dicarboxylic acid bis(4-aminophenyl)ester with a purity of 99.4% (areal percentage by high performance liquid chromatography) as pale gray powder.

Herein, biphenyl-4,4′-dicarboxylic acid bis(4-aminophenyl)ester was a novel compound shown by the following physical properties.

Melting point; 248 to 251° C.

¹H -NMR(DMSO-d₆,δ(ppm)); 5.10(4H,brs,NH2), 6.40-6.66(4H,m), 6.90-6.98(4H,m), 7.80-8.08(4H,m), 8.01-8.25(4H,m)

### Example A-3

### (B) Synthesis of biphenyl-4,4′-dicarboxylic acid bis(4-aminophenyl)ester

To a flask having an inner volume of 500 ml and equipped with a stirring device, a thermometer and a reflux condenser were added 30.0 g (61.9 mmol) of biphenyl-4,4′-dicarboxylic acid bis(4-nitrophenyl)ester prepared in the same manner as in Example A-1,330 ml of N,N-dimethylformamide and 6.29 g (52 mg calculated as palladium atom, 78 mg calculated as platinum atom) of 2% by mass palladium-3% by mass platinum/carbon (UKH-10 manufactured by N.E. CHEMCAT Corporation, 49.8% by mass wet product). The flask was equipped with a balloon charged with hydrogen gas and reaction was carried out at 80° C. for 5 hours with stirring. After completion of the reaction, the reaction solution was cooled to 60° C. and then filtered. 300 ml of water was added to the filtrate, and the mixture was stirred at 25° C. for 0.5 hour. The precipitated crystal was filtered and the filtered residue was washed with 50 ml of N,N-dimethylformamide, 100 ml of water and 100 ml of methanol in order, and then dried to obtain 24.8 g (58.4 mmol) of biphenyl-4,4′-dicarboxylic acid bis(4-aminophenyl)ester with a purity of 96.7% (areal percentage by high performance liquid chromatography) as skin color powder (Reaction yield; 94% on the basis of biphenyl-4,4′-dicarboxylic acid bis(4-nitrophenyl)ester).

### Comparative Reference Example A-1

In order to compare physical properties of polyimide, terephthalic acid bis(4-aminophenyl)ester was synthesized in the same manner as described in Patent Document 1 as a diamine compound.

### Synthesis of Polyimide and Evaluation of Film

Next, Examples of the polyimide of the present invention will be explained. Herein, measured values shown in Examples and Comparative Examples were measured in the following methods

1) Solution Viscosity

The solution viscosity of a polyamic acid was measured using a TV-20 viscometer (cone-plate type) manufactured by Toki Sangyo Co., Ltd., with a cone rotor 3°×R14, at 25° C. in the range of 0.5 to 10 rpm.

2) Tensile Test

A film was punched in an IEC450 standard dumbbell shape to give a test piece. An initial elastic modulus, breaking strength and elongation at break were measured under conditions of 30 mm for a space of chucks and a tensile rate of 2 mm/min by the use of TENSILON manufactured by ORIENTEC Company Limited.

3) Measurement of Solid Viscoelasticity

The film was cut in a shape of strip of 2 cm×2 mm to give a test piece, and the solid viscoelasticity was measured in the tensile mode using RSAII manufactured by TA Instruments. The measuring was carried out at 10 Hz while the test piece was heated in a stream of nitrogen from room temperature to the limit temperature at a rate of 3° C./step. The elastic modulus at 400° C. was determined from the obtained E′ curve. A glass transition temperature (Tg) was determined from the maximum of the E″ curve.

4) Water Absorption Percentage

A film of 15 cm×15 cm was vacuum-dried at 150° C. for 2 hours and the dry weight W₀ was measured. Thereafter, the film was immersed in water at 23° C. and allowed to stand for 24 hours. Water adhered on the film surface was wiped out with filter paper, the weight W₁ after absorption was measured and the water absorption percentage was determined from Equation (1).

Water Absorption (%) (W₁−W₀)/W₀×100 Equation (1)

5) Coefficient of Hygroscopic Expansion (CHE)

Shallow lines in a lattice shape were put at intervals of about 1 cm in a region of 5 cm×5 cm of the film by the use of a cutter and the resulting material was vacuum-dried at 150° C. for 2 hours. An interval Lo between lattice points of this dry film was recorded in the unit of 1 μm using MM-40 of a measuring microscope manufactured by Nikon. Thereafter, the film was immersed in water at 23° C. and allowed to stand for 24 hours. Water adhered on the film surface was wiped out with filter paper, an interval L₁ between lattice points after absorption was recorded in the same manner, and the hygroscopic expansion coefficient was calculated according to Equation (2). An average was taken from 15 values.

Coefficient of Hygroscopic Expansion (ppm/RH %)=(L₁−L₀)/L₀/100×10⁶ Equation (2)

6) Coefficient of Thermal Expansion (CTE)

The film was cut in a shape of strip of 10 mm length to give a test piece and heated to 400° C. at a rate of 5° C./min with a load of 5 g using TMA-50 manufactured by Shimadzu Corporation. An average thermal expansion coefficient at 50° C. to 200° C. was determined from the obtained TMA curve.

7) Thermogravimetric Analysis

Using TGA-50 manufactured by Shimadzu Corporation, the film was heated at a rate of 10° C./min in a nitrogen atmosphere. 5% weight loss temperature (Td₅) was determined from the obtained thermogravimetric loss curve.

8) APBP Unit Weight %

Weight % of APBP unit was determined from Equation (3), for example, when acid dianhydride and diamine were respectively consisting of two components.

APBP Unit Weight %=(M₁A₁B₁+M₃A₂B₁)/(M₁A₁B₁+M₂A₁B₂+M₃A₂B₁+M₄A₂B₂)×100 Equation (3)

Herein, in feeding of monomers, mol fraction of the first component of the acid dianhydride occupied in the total acid dianhydride component is denoted as A₁, while mol fraction of the second component of the acid dianhydride is denoted as A₂. Furthermore, mol fraction of APBP occupied in the total diamine component is denoted as B_{1,} while mol fraction of the second component of diamine is denoted as B₂. In the final composition of the polyimide, the molecular weight of the constituent unit consisting of the first component of the acid dianhydride and APBP is denoted as M₁, the molecular weight of the constituent unit consisting of the first component of the acid dianhydride and the second component of diamine is denoted as M₂, the molecular weight of the constituent unit consisting of the second component of the acid dianhydride and APBP is denoted as M₃, and the molecular weight of the constituent unit consisting of the second component of the acid dianhydride and the second component of diamine is denoted as M₄. For example, when the first and second components of the acid dianhydride, and the second component of diamine are respectively s-BPDA, PMDA and PPD, M₁, M₂, M₃ and M₄ are respectively 682.63, 366.33, 606.54 and 290.23.

Even though each component is not composed of two components, weight % of APBP unit can be determined by increasing or decreasing the number of terms in the Equation based on the same idea. Herein, the APBP unit refers to a constituent unit consisting of acid dianhydride and APBP in the final composition of the polyimide. For example, the constituent unit consisting of s-BPDA and APBP is represented by the formula (4).

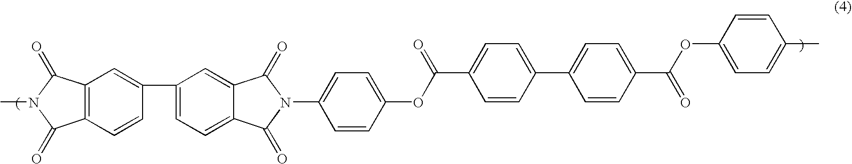

Herein, weight fraction of APB unit was also determined in the same idea.

Diamine Compound:

Biphenyl-4,4′-dicarboxylic acid bis(4-aminophenyl)ester (hereinafter abbreviated as APBP) synthesized in Example A-2 was used.

Terephthalic acid bis(4-aminophenyl)ester (hereinafter abbreviated as APB) synthesized in Comparative Reference Example A-1 was used.

Following abbreviations will be used.

_{S}-BPDA: 3,3′,4,4′-biphenyltetracarboxylic dianhydride

PPD: p-phenylenediamine

PMDA: pyromellitic dianhydride

ODPA: bis(3,4-dicarboxyphenyl)ether dianhydride

DMAc: N,N-dimethylacetamide

### Example B-1

### Preparation of s-BPDA/APBP Film

5.000 g of APBP was dissolved in 38.6 g of N,N-dimethylacetamide, to which 3,3′,4,4′-biphenyltetracarboxylic dianhydride (s-BPDA) was gradually added to be equimolar to APBP with stirring, and the mixture was reacted to obtain a polyamic acid solution in which the monomer feed amount was 18 weight %. The viscosity of the solution was 150 Pa·s. The polyamic acid solution was cast on a glass plate such that the final film thickness was about 30 μm and dried at 120° C. for 20 minutes. The resulting film was peeled off, fixed to a pin tenter, heated at 180° C. and 210° C., respectively for 5 minutes each, and then heated from 270° C. to 450° C. for 9 minutes to obtain a polyimide film. The composition and viscosity of the polyamic acid solution are shown in Table 1, and properties of the polyimide film are shown in Table 2.

### Example B-2

### Preparation of s-BPDA/APBP/PPD(3/2/1) Thermally Imidized Film

6.000 g of APBP and 0.764 g of PPD were dissolved in 59.22 g of DMAc, to which s-BPDA was gradually added to be equimolar to the diamine component with stirring, and the mixture was reacted to obtain a polyamic acid solution in which the monomer feed amount was 18 weight %. The viscosity of the solution was 180 Pa·s. The polyamic acid solution was cast on a glass plate such that the final film thickness was about 20 μm and dried at 120° C. for 30 minutes. The resulting film was peeled off, fixed to a pin tenter, heated at 180° C. and 210° C., respectively for 5 minutes each, and then heated from 270° C. to 450° C. for 9 minutes to obtain a polyimide film. The composition and viscosity of the polyamic acid solution are shown in Table 1, and properties of the polyimide film are shown in Table 2.

### Example B-3

### Preparation of s-BPDA/APBP/PPD (3/2/1) Chemically Imidized Film

To the polyamic acid solution obtained in Example B-2 were added a DMAc solution of 1 equivalent of acetic anhydride and a DMAc solution of 0.5 equivalent of of isoquinoline, both based on the carboxylic acid of the polyamic acid, at −10° C., and the mixture was degassed. The amount of DMAc was such that the polyamic acid was 9 weight %. The obtained 9 weight % polyamic acid solution was cast on a glass plate such that the final film thickness was about 20 μm and dried at 120° C. for 5 minutes. The resulting film was peeled off, fixed to a pin tenter, heated at 180° C. and 210° C., respectively for 5 minutes each, and then heated from 270° C. to 450° C. for 9 minutes to obtain a polyimide film. The composition and viscosity of the polyamic acid solution are shown in Table 1, and properties of the polyimide film are shown in Table 2.

### Comparative Example B-1

### Preparation of s-BPDA/APB Film

5.000 g of APB was dissolved in 42.06 g of DMAc, to which s-BPDA was gradually added to be equimolar to APB with stirring, and the mixture was reacted to obtain a polyamic acid solution in which the monomer feed amount was 18 weight %. This solution had an extremely high viscosity. The solution was diluted to 9 weight % with DMAc to obtain a solution of 7 Pa·s. The solution was cast on a glass plate such that the final film thickness was about 30 μm and dried at 120° C. for 30 minutes. The resulting film was peeled off, fixed to a pin tenter, heated at 180° C. and 210° C., respectively for 5 minutes each, and then heated from 270° C. to 450° C. for 9 minutes to obtain a polyimide film. The composition and viscosity of the polyamic acid solution are shown in Table 1, and properties of the polyimide film are shown in Table 2.

TABLE 1

| | | | | | | | polyamic acid | |
|---|---|---|---|---|---|---|---|---|
| | | | | APBP | APBP unit | | Solution | |
| | | | | or | or | | viscosity Pa · s | |
| | acid | | s-BPDA¹⁾ | APB²⁾ | APB unit | | | after |
| | dianhydride | diamine | mol % | mol % | wt % | Solvent | 18% | diluted |
| Ex. B-1 | s-BPDA | APBP | 100 | 100 | 100 | DMAc | 150 | — |
| Ex. B-2 | ↑ | APBP/ | ↑ | 67 | 79 | ↑ | 180 | — |
| | | PPD | | | | | | |
| Ex. B-3 | ↑ | ↑ | ↑ | ↑ | ↑ | ↑ | ↑ | — |
| Comp. Ex. | ↑ | APB | ↑ | 100 | 100 | ↑ | high | 9%, |
| B-1 | | | | | | | viscosity | 7 Pa · s |

¹⁾s-BPDA mol % in acid dianhydride(s)
²⁾APBP or APB mol % in diamine(s)

TABLE 2

| | | initial | | | water | | | | | |
|---|---|---|---|---|---|---|---|---|---|---|
| | film | elastic | breaking | elongation | absorption | CHE | | E′ @ | | |
| | thickness | modulus | strength | at break | percentage | ppm/ | CTE | 400° C. | Tg | Td5 |
| | μm | GPa | MPa | % | % | RH % | ppm/K | GPa | ° C. | ° C. |
| Ex. B-1 | 29 | 6.4 | 270 | 18 | 0.56 | 3.5 | 8.0 | 1.57 | 236 | 533 |
| Ex. B-2 | 22 | 7.1 | 270 | 17 | 0.68 | 4.0 | 8.8 | 1.07 | 241 | 542 |
| Ex. B-3 | 21 | 9.8 | 400 | 14 | 0.76 | 2.9 | 5.7 | 1.58 | 240 | 535 |
| Comp. | 30 | 6.0 | 210 | 11 | 1.02 | 7.4 | 8.8 | 2.04 | 243 | 518 |
| Ex. B-1 | | | | | | | | | | |

### Example B-4

### Preparation of s-BPDA/APBP/PPD(2/1/1) Film

5.000 g of APBP and 1.273 g of PPD were dissolved in 60.16 g of DMAc, to which s-BPDA was gradually added to be equimolar to the diamine component with stirring, and the mixture was reacted to obtain a polyamic acid solution in which the monomer feed amount was 18 weight %. The viscosity of the solution was 150 Pa·s. The polyamic acid solution was cast on a glass plate such that the final film thickness was about 30 μm and dried at 120° C. for 20 minutes. The resulting film was peeled off, fixed to a pin tenter, heated at 180° C. and 210° C., respectively for 5 minutes each, and then heated from 270° C. to 450° C. for 9 minutes to obtain a polyimide film. The composition and viscosity of the polyamic acid solution are shown in Table 3, and properties of the polyimide film are shown in Table 4.

### Example B-5

### Preparation of s-BPDA/APBP/PPD (10/3/7) Film

3.000 g of APBP and 1.783 g of PPD were dissolved in 53.34 g of DMAc, to which s-BPDA was gradually added to be equimolar to the diamine component with stirring, and the mixture was reacted to obtain a polyamic acid solution in which the monomer feed amount was 18 weight %. The viscosity of the solution was 150 Pa·s. The polyamic acid solution was cast on a glass plate such that the final film thickness was about 30 μm and dried at 120° C. for 20 minutes. The resulting film was peeled off, fixed to a pin tenter, heated at 180° C. and 210° C., respectively for 5 minutes each, and then heated from 270° C. to 450° C. for 9 minutes to obtain a polyimide film. The composition and viscosity of the polyamic acid solution are shown in Table 3, and properties of the polyimide film are shown in Table 4.

### Example B-6

### Preparation of s-BPDA/APBP/PPD (10/1/9) Film

2.000 g of APBP and 4.586 g of PPD were dissolved in 93.16 g of DMAc, to which s-BPDA was gradually added to be equimolar to the diamine component with stirring, and the mixture was reacted to obtain a polyamic acid solution in which the monomer feed amount was 18 weight %. The viscosity of the solution was 150 Pa·s. The polyamic acid solution was cast on a glass plate such that the final film thickness was about 30 μm and dried at 120° C. for 20 minutes. The resulting film was peeled off, fixed to a pin tenter, heated at 180° C. and 210° C., respectively for 5 minutes each, and then heated from 270° C. to 450° C. for 9 minutes to obtain a polyimide film. The composition and viscosity of the polyamic acid solution are shown in Table 3, and properties of the polyimide film are shown in Table 4.

### Comparative Example B-2

### Preparation of s-BPDA/PPD Film

5.000 g of PPD was dissolved in 84.8 g of DMAc, to which s-BPDA was gradually added to be equimolar to PPD with stirring, and the mixture was reacted to obtain a polyamic acid solution in which the monomer feed amount was 18 weight %. The viscosity of the solution was 150 Pa·s. The polyamic acid solution was cast on a glass plate such that the final film thickness was about 30 μm and dried at 120° C. for 20 minutes. The resulting film was peeled off, fixed to a pin tenter, heated at 180° C. and 210° C., respectively for 5 minutes each, and then heated from 270° C. to 450° C. for 9 minutes to obtain a polyimide film. The composition and viscosity of the polyamic acid solution are shown in Table 3, and properties of the polyimide film are shown in Table 4.

TABLE 3

| | | | | | | | polyamic acid |
|---|---|---|---|---|---|---|---|
| | | | | | APBP | | solution |
| | acid | | s-BPDA¹⁾ | APBP²⁾ | unit | | viscosity Pa · s |
| | dianhydride | diamine | mol % | mol % | wt % | Solvent | 18% |
| Ex. B-4 | s-BPDA | APBP/ | 100 | 50 | 65 | DMAc | 150 |
| | | PPD | | | | | |
| Ex. B-5 | ↑ | ↑ | ↑ | 30 | 44 | ↑ | ↑ |
| Ex. B-6 | ↑ | ↑ | ↑ | 10 | 17 | ↑ | ↑ |
| Comp. | ↑ | PPD | ↑ | 0 | 0 | ↑ | 150 |
| Ex. B-2 | | | | | | | |

¹⁾s-BPDA mol % in acid dianhydride(s)
²⁾APBP mol % in diamine(s)

TABLE 4

| | | initial | | | water | | | | | |
|---|---|---|---|---|---|---|---|---|---|---|
| | film | elastic | breaking | elongation | absorption | | | E′ @ | | |
| | thickness | modulus | strength | at break | percentage | CHE | CTE | 400° C. | Tg | Td5 |
| | μm | GPa | MPa | % | % | ppm/RH % | ppm/K | GPa | ° C. | ° C. |
| Ex. B-4 | 27 | 7.2 | 280 | 17 | 0.72 | 4.8 | 6.0 | 1.35 | 260 | 552 |
| Ex. B-5 | 34 | 7.3 | 290 | 17 | 0.84 | 6.2 | 6.6 | 1.32 | 282 | 555 |
| Ex. B-6 | 33 | 7.5 | 300 | 12 | 1.22 | 9.2 | 10 | 1.47 | 309 | 590 |
| Comp. | 30 | 8.0 | 380 | 22 | 1.44 | 11.0 | 14 | | | |
| Ex. B-2 | | | | | | | | | | |

### Reference Example B-1

### Preparation of PMDA/APBP Film

6.000 g of APBP was dissolved in 37.20 g of DMAc, to which pyromellitic dianhydride (PMDA) was gradually added to be equimolar to APBP with stirring, and the mixture was reacted to obtain a polyamic acid solution in which the monomer feed amount was 18 weight %. The solution had an extremely high viscosity. Immediately after the solution was diluted to 14 weight % with DMAc, the viscosity of the solution was 18 Pa·s. When the solution was allowed to stand for one day, it became gel state so that it was unable to make a film.

### Example B-7

### Preparation of PMDA/s-BPDA/APBP(2/1/3) Film

5.000 g of APBP was dissolved in 37.20 g of DMAc, to which PMDA and s-BPDA were gradually added to be equimolar to APBP with stirring, and the mixture was reacted to obtain a polyamic acid solution in which the monomer feed amount was 14 weight % The molar ratio of PMDA and s-BPDA was 2:1. The viscosity of the solution was 190 Pa·s. The polyamic acid solution was cast on a glass plate such that the final film thickness was about 30 μm and dried at 120° C. for 20 minutes. The resulting film was peeled oft fixed to a pin tenter, heated at 180° C. and 210° C., respectively for 5 minutes each, and then heated from 270° C. to 450° C. for 9 minutes to obtain a polyimide film. The composition and viscosity of the polyamic acid solution are shown in Table 5, and properties of the polyimide film are shown in Table 6.

### Example B-8

### Preparation of ODPA/APBP Film

5.000 g of APBP was dissolved in 39.42 g of DMAc, to which ODPA was gradually added to be equimolar to APBP with stirring, and the mixture was reacted to obtain a polyamic acid solution in which the monomer feed amount was 14 weight %. The viscosity of the solution was 80 Pa·s. The polyamic acid solution was cast on a glass plate such that the final film thickness was about 30 μm, and heated at 120° C. for 20 minutes and at 180° C. for 5 minutes. The resulting film was peeled off from the glass plate, fixed to a pin tenter, heated at 210° C. for 5 minutes, and then heated from 270° C. to 450° C. for 9 minutes to obtain a polyimide film. The composition and viscosity of the polyamic acid solution are shown in Table 5, and properties of the polyimide film are shown in Table 6.

### Example B-9

### Preparation of ODPA/s-BPDA/APBP(2/1/3) Film

5.000 g of APBP was dissolved in 38.84 g of DMAc, to which ODPA and s-BPDA were gradually added to be equimolar to APBP with stirring, and the mixture was reacted to obtain a polyamic acid solution in which the monomer feed amount was 14 weight %. The molar ratio of ODPA and s-BPDA was 2:1. The viscosity of the solution was 190 Pa·s. The polyamic acid solution was cast on a glass plate such that the final film thickness was about 30 μm, and heated at 120° C. for 20 minutes and at 180° C. for 5 minutes. The resulting film was peeled off from the glass plate, fixed to a pin tenter, heated at 210° C. for 5 minutes, and then heated from 270° C. to 450° C. for 9 minutes to obtain a polyimide film. The composition and viscosity of the polyamic acid solution are shown in Table 5, and properties of the polyimide film are shown in Table 6.

TABLE 5

| | | | | | | | polyamic acid |
|---|---|---|---|---|---|---|---|
| | | | | | APBP | | solution |
| | acid | | s-BPDA¹⁾ | APBP²⁾ | unit | | viscosity Pa · s |
| | dianhydride | diamine | mol % | mol % | wt % | Solvent | 14% |
| Ref. | PMDA | APBP | 0 | 100 | 100 | DMAc | 18 |
| Ex. B-1 | | | | | | | |
| Ex. B-7 | PMDA/ | ↑ | 33 | 100 | 100 | ↑ | 190 |
| | s-BPDA | | | | | | |
| Ex. B-8 | ODPA | ↑ | 0 | 100 | 100 | ↑ | 80 |
| Ex. B-9 | ODPA/ | ↑ | 33 | 100 | 100 | ↑ | 190 |
| | s-BPDA | | | | | | |

¹⁾s-BPDA mol % in acid dianhydride(s)
²⁾APBP mol % in diamine(s)

TABLE 6

| | | water absorption |
|---|---|---|
| | film thickness | percentage |
| | μm | % |
| Example B-7 | 31 | 0.50 |
| Example B-8 | 25 | 0.60 |
| Example B-9 | 33 | 0.69 |

### Synthesis Examples of Diamine Compounds According to Production Method 11-1

Next, Synthesis Examples of the diamine compounds according to Production Method II-1 will be described in detail. Analysis conditions by high performance liquid chromatography used in each Example and Reference Example are as follows.

Instrument Type: Shimadzu high performance liquid chromatography, LC-10A

Column: YMC-PackPro,C₁₈,s·5 μm,4.6I.D*150 mm

Eluent: water/acetonitrile=1.2/1.8 (volume ratio)

pH: 7.0 (acetic acid (0.1 ml/l) added to have pH of 7.0 with triethylamine)

Flow rate: 1.0 ml/min

Column oven temperature: 40° C.

Detection wavelength: 254 nm

### Reference Example C-1

### Synthesis of biphenyl-4,4′-dicarboxylic acid diphenyl ester

In a flask having an inner volume of 1,000 ml and equipped with a stirring device and a thermometer, 41.5 g (0.410 mol) of triethylamine, 680 ml of tetrahydrofuran and 29.7 g (0.316 mol) of phenol were mixed. To this mixed solution was slowly added 40.0 g (0.143 mol) of 4,4′-biphenyldicarbonyl chloride while maintaining the liquid temperature at 25° C. or less, and then the resulting mixture was stirred at 25° C. for 15 hours. After completion of the reaction, the reaction solution was filtered. 1,300 ml of water was added to the resulting solid. The mixture was stirred at 25° C. for 1 hour and then filtered. The resulting solid was washed with 800 ml of water and 60 ml of tetrahydrofuran in order, and then dried to obtain 51.4 g of biphenyl-4,4′-dicarboxylic acid diphenyl ester as a white solid (Isolation yield: 91% on the basis of 4,4′-biphenyldicarbonyl chloride).

Physical properties of the obtained biphenyl-4,4′-dicarboxylic acid diphenyl ester were as follows.

¹H-NMR(300 MHz,THF-d₈,δ(ppm)); 7.15-7.30(6H,m),7.33-7.50(4H,m),7.88-8.01(4H,m),8.22-8.35(4H,m)

### Reference Example C-2

### Synthesis of biphenyl-4,4′-dicarboxylic acid bis(4-nitrophenyl)ester

Biphenyl-4,4′-dicarboxylic acid bis(4-nitrophenyl)ester was synthesized in the same manner as in Example A-1.

### Reference Example C-3

### Synthesis of biphenyl-4,4′-dicarboxylic acid bis(2-chlorophenyl)ester

In a flask having an inner volume of 500 ml and equipped with a stirring device and a thermometer, 20.7 g (205 mmol) of triethylamine, 340 ml of tetrahydrofuran and 20.3 g (158 mmol) of 2-chlorophenol were mixed. To this mixed solution was added 20.0 g (71.7 mmol) of 4,4′-biphenyldicarbonyl chloride while maintaining the liquid temperature at 30° C. or less, and then the resulting mixture was stirred at 25° C. for 15 hours. After completion of the reaction, the reaction solution was filtered and 670 ml of water was added to the resulting solid. The mixture was stirred at 25° C. for 1 hour and then filtered. The resulting solid was washed with 800 ml of water and 60 ml of tetrahydrofuran in order and then dried to obtain 25.8 g of biphenyl-4,4′-dicarboxylic acid bis(2-chlorophenyl)ester as a white solid (Isolation yield: 78% on the basis of 4,4′-biphenyldicarbonyl chloride).

Physical properties of the resulting biphenyl-4,4′-dicarboxylic acid bis(2-chlorophenyl)ester were as follows.

¹H-NMR(300 MHz,THF-d₈,δ(ppm)); 7.25-7.32(2H,m),7.37-7.39(4H,m),7.52-7.55(2H,m),7.94-7.88(4H,m),8.31-8.35(4H,m)

### Example C-1

### Synthesis of biphenyl-4,4′-dicarboxylic acid bis(4-aminophenyl)ester

In a flask having an inner volume of 25 ml and equipped with a stirring device and a thermometer, 1.58 g (4.00 mmol) of biphenyl-4,4′-dicarboxylic acid diphenyl ester synthesized in Reference Example C-1, 1.31 g (12.0 mmol) of 4-aminophenol, 40 ml of N,N-dimethylformamide and 0.304 g (2.00 mmol) of 1,8-diazabicyclo[5,4,1]-7-undecene were mixed under an argon gas stream, and the mixed solution was stirred at a liquid temperature of 93° C. for 3 hours. After completion of the reaction, the reaction solution was cooled to 25° C. or less, 40 ml of water was added thereto and then the mixture was filtered. The resulting solid was washed with 10 ml of water and 10 ml of methanol in order, and then dried to obtain 1.62 g of a light brown solid.

The light brown solid was analyzed by high performance liquid chromatography and as a result, the ratio of biphenyl-4,4′-dicarboxylic acid bis(4-aminophenyl)ester (desired product) to biphenyl-4,4′-dicarboxylic acid 4-(4-aminophenyl)ester 4′-phenyl ester (precursor of the desired product) was 87:13 (areal percentage). Furthermore, this solid was quantitatively analyzed by high performance liquid chromatography and as a result, the amount of biphenyl-4,4′-dicarboxylic acid bis(4-aminophenyl)ester was 1.41 g (Yield: 83% on the basis of biphenyl-4,4′-dicarboxylic acid diphenyl ester).

Physical properties of biphenyl-4,4′-dicarboxylic acid bis(4-aminophenyl)ester were as follows.

¹H-NMR(300 MHz,DMSO-d₆,δ(ppm)); 5.10(4H,brs,NH2),6.40-6.66(4H,m) ,6.90-6.98(4H,m), 7.80-8.08(4H,m),9.01-8.25 (4H,m)

Physical properties of biphenyl-4,4′-dicarboxylic acid 4-(4-aminophenyl)ester 4′-phenyl ester were as follows.

¹H-NMR(300 MHz,DMSO-d₆,δ(ppm)); 5.10(2H,brs,NH2),6.60-6.65(2H,m),6.88-6.70(2H,m),7.28-7.41(3H,m),7.42-7.58 (2H,m),7.95-8.10(4H,m),8.15-8.33(4H,m)

### Example C-2

### Synthesis of biphenyl-4,4′-dicarboxylic acid bis(4-aminophenyl)ester

All operations were carried out in the same manner as in Example C-1, except that the amount of 4-aminophenol used in Example C-1 was changed to 2.18 g (20.0 mmol), to obtain 1.62 g of a light brown solid. The light brown solid was analyzed by high performance liquid chromatography and as a result, biphenyl-4,4′-dicarboxylic acid bis(4-aminophenyl)ester (desired product) and biphenyl-4,4′-dicarboxylic acid 4-(4-aminophenyl)ester 4-phenyl ester (precursor of the desired product) were prepared in the ratio of 95:5 (areal percentage). Furthermore, this solid was quantitatively analyzed by high performance liquid chromatography and as a result, biphenyl-4,4′-dicarboxylic acid bis(4-aminophenyl)ester was contained in an amount of 1.52 g (Yield: 90% on the basis of biphenyl-4,4′-dicarboxylic acid diphenyl ester).

### Example C-1-2

### Synthesis of biphenyl-4,4′-dicarboxylic acid bis(4-aminophenyl)ester

In a flask having an inner volume of 25 ml and equipped with a stirring device, a thermometer and a dropping funnel, 1.58 g (4.00 mmol) of biphenyl-4,4′-dicarboxylic acid diphenyl ester synthesized in Reference Example C-1, 1.31 g (12.0 mmol) of 4-aminophenol, 10 ml of 1,2-dichlorobenzene and 0.122 g (0.801 mmol) of 1,8-diazabicyclo[5,4,0]-7-undecene were mixed, and the resulting mixture was stirred for 1 hour while the liquid temperature was maintained at 100° C. Subsequently, to this mixed solution was added 3 ml of 1,2-dichlorobenzene. At a reaction temperature of 95 to 99° C. and a reaction pressure of 9.3 kPa, an operation for removing the solvent slowly in vacuo was repeatedly carried out 5 times in total. After completion of the reaction, the reaction solution was cooled to 25° C. and then filtered, and the resulting solid was dried to obtain 2.14 g of brown powder.

The resulting powder was analyzed by high performance liquid chromatography (absolute calibration curve method) and as a result, biphenyl-4,4′-dicarboxylic acid bis(4-aminophenyl)ester, a desired product, was contained in an amount of 1.26 g (Yield: 74% on the basis of biphenyl-4,4′-dicarboxylic acid diphenyl ester). Furthermore, in the powder biphenyl-4,4′-dicarboxylic acid 4-(4-aminophenyl)ester-4′-phenyl ester (precursor of the desired product) was contained in an amount of 0.38 g (Yield: 23% on the basis of biphenyl-4,4′-dicarboxylic acid diphenyl ester).

Physical properties of the resulting powder were as follows.

Biphenyl-4,4′-dicarboxylic acid bis(4-aminophenyl)ester;

¹H-NMR(300 MHz,DMSO-d₆,δ(ppm));

5.10(4H,brs),6.40-6.66(4H,m),6.90-6.98(4H,m),7.80-8.08(4H,m),8.01-8.25(4H, m)

Biphenyl-4,4′-dicarboxylic acid 4-(4-aminophenyl)ester 4′-phenyl ester;

¹H-NMR(300 MHz,DMSO-d₆,δ(ppm));

5.10(2H,brs),6.60-6.65(2H,m),6.88-6.70(2H,m),7.28-7.41(3H,m),7.42-7.58(2H, m),7.95-8.10(4H,m),8.15-8.33(4H,m)

### Example C-3

### Synthesis of biphenyl-4,4′-dicarboxylic acid bis(4-aminophenyl)ester

In a flask having an inner volume of 500 ml and equipped with a stirring device and a thermometer, 10.5 g (109 mmol) of sodium t-butoxide was mixed with 250 ml of tetrahydrofuran at room temperature under an argon gas stream and subsequently 12.4 g (114 mmol) of 4-aminophenol was slowly added thereto, and the resulting mixture was stirred for 30 minutes. 24.0 g (49.5 mmol) of biphenyl-4,4′-dicarboxylic acid bis(4-nitrophenyl)ester synthesized in Reference Example C-2 was slowly added to the resulting mixed solution at room temperature and the mixture was stirred for 2 hours. After completion of the reaction, the reaction solution was filtered, and the resulting solid was washed with 30 ml of tetrahydrofuran and 50 ml of methanol in order, and then dried to obtain 17.8 g of a light yellow solid. The light yellow solid was quantitatively analyzed by high performance liquid chromatography and as a result, the amount of biphenyl-4,4′-dicarboxylic acid bis(4-aminophenyl)ester was 16.6 g (Yield: 79% on the basis of biphenyl-4,4′-dicarboxylic acid bis(4-nitrophenyl)ester).

### Example C-4

### Synthesis of biphenyl-4,4′-dicarboxylic acid bis(4-aminophenyl)ester

In a flask having an inner volume of 25 ml and equipped with a stirring device and a thermometer, 1.75 g (3.78 mmol) of biphenyl-4,4′-dicarboxylic acid bis(2-chlorophenyl)ester synthesized in Reference Example C-3, 2.06 g (18.9 mmol) of 4-aminophenol, 14.2 ml of N,N-dimethylformamide and 0.115 g (0.755 mmol) of 1,8-diazabicyclo[5,4,0]-7-undecene were mixed under an argon gas stream. This mixed solution was stirred at a liquid temperature of 90° C. for 6.5 hours. After completion of the reaction, the reaction solution was cooled to 25° C. or less and then filtered. The resulting solid was washed with 2 ml of N,N-dimethylformamide and 2 ml of methanol in order, and then dried to obtain 1.07 g of a light yellow solid.

The light yellow solid was analyzed by high performance liquid chromatography and as a result, biphenyl-4,4′-dicarboxylic acid bis(4-aminophenyl)ester (desired product) and biphenyl-4,4′-dicarboxylic acid 4-(4-aminophenyl)ester 4′-(2-chlorophenyl)ester (precursor of the desired product) were prepared in the ratio of 99.6:0.4 (areal percentage). Furthermore, this solid was quantitatively analyzed by high performance liquid chromatography and as a result, biphenyl-4,4′-dicarboxylic acid bis(4-aminophenyl)ester was contained in an amount of 1.06 g (Yield: 66% on the basis of biphenyl-4,4′-dicarboxylic acid bis(2-chlorophenyl)ester).

Meanwhile, the filtrate obtained after filtering the aforementioned reaction solution was analyzed by high performance liquid chromatography and as a result, the ratio of biphenyl-4,4′-dicarboxylic acid bis(4-aminophenyl)ester (desired product) to biphenyl-4,4′-dicarboxylic acid 4-(4-aminophenyl)ester 4′-(2-chlorophenyl)ester (precursor of the desired product) was 97.6:2.4 (areal percentage). Furthermore, this filtrate was quantitatively analyzed by high performance liquid chromatography and as a result, biphenyl-4,4′-dicarboxylic acid bis(4-aminophenyl)ester was contained in an amount of 0.4 g (Yield: 24% on the basis of biphenyl-4,4′-dicarboxylic acid bis(2-chlorophenyl)ester).

### Example C-5

### Synthesis of biphenyl-4,4′-dicarboxylic acid bis(4-chlorophenyl)ester

In a flask having an inner volume of 500 ml and equipped with a stirring device, a thermometer and a dropping funnel, 20.7 g (205 mmol) of triethylamine, 340 ml of tetrahydrofuran and 20.3 g (158 mmol) of 4-chlorophenol were mixed, and 20.0 g (71.7 mmol) of 4,4′-biphenyldicarbonyl chloride was slowly added thereto while maintaining the liquid temperature at 10° C. or less, and the resulting mixture was reacted at 25° C. for 19 hours. After completion of the reaction, the reaction solution was filtered and the filtered residue was mixed with 333 ml of water, and was stirred at 25° C. for 1 hour The mixture was filtered again, and the resulting solid was washed with 800 ml of water and 60 ml of tetrahydrofuran in order, and then dried to obtain 27.5 g of biphenyl-4,4′-dicarboxylic acid bis(4-chlorophenyl)ester as white powder (Isolation yield: 81% on the basis of 4,4′-biphenyldicarbonyl chloride).

The resulting biphenyl-4,4′-dicarboxylic acid bis(4-chlorophenyl)ester was a novel compound shown by the following physical properties.

¹H-NMR(300 MHz,DMSO-d₆,δ(ppm)); 7.31-7.47(4H,m),7.49-7.63(4H,m),7.98-8.10(4H,m),8.30-8.33(4H,m)

### Example C-6

### Synthesis of biphenyl-4,4′-dicarboxylic acid bis(4-aminophenyl)ester

In a flask having an inner volume of 25 ml and equipped with a stirring device and a thermometer, 1.85 g (4.00 mmol) of biphenyl-4,4′-dicarboxylic acid bis(4-chlorophenyl)ester synthesized in Example C-5, 1.31 g (12.0 mmol) of 4-aminophenol, 15 ml of N,N-dimethylformamide and 0.122 g (0.880 mmol) of 1,8-diazabicyclo[5,4,0]-7-undecene were mixed under an argon gas stream. This mixed solution was heated and stirred at a liquid temperature of 92° C. for 6.5 hours. After completion of the reaction, the reaction solution was cooled to 25° C. and 15 ml of water was added thereto, and the mixture was filtered. The resulting solid was washed with 15 ml of water and 10 ml of methanol in order, and then dried to obtain 1.42 g of a brown solid.

The brown solid was analyzed by high performance liquid chromatography and as a result, biphenyl-4,4′-dicarboxylic acid bis(4-aminophenyl)ester (desired product) and biphenyl-4,4′-dicarboxylic acid 4-(4-aminophenyl)ester 4′-(4-chlorophenyl)ester (intermediate of the desired product) were prepared in the ratio of 94:6 (areal percentage). Furthermore, the brown solid was quantitatively analyzed by high performance liquid chromatography and as a result, biphenyl-4,4′-dicarboxylic acid bis(4-aminophenyl)ester was contained in an amount of 1.30 g (Yield: 76% on the basis of biphenyl-4,4′-dicarboxylic acid bis(4-chlorophenyl)ester).

### Example C-7

### Synthesis of biphenyl-4,4′-dicarboxylic acid bis(4-aminophenyl)ester

All operations were carried out in the same manner as in Example C-6, except that the base used in Example C-6 was changed from 1,8-diazabicyclo[5,4,0]-7-undecene to potassium carbonate, its amount used was changed to 1.11 g (8.00 mmol) and the reaction time was changed to 4 hours, to obtain 1.43 g of a light brown solid. The resulting solid was analyzed by high performance liquid chromatography and as a result, biphenyl-4,4′-dicarboxylic acid bis(4-aminophenyl)ester (desired product) and biphenyl-4,4′-dicarboxylic acid 4-(4-aminophenyl)ester 4′-(4-chlorophenyl)ester (intermediate of the desired product) were prepared in the ratio of 96:4 (areal percentage). Furthermore, this solid was quantitatively analyzed by high performance liquid chromatography and as a result, biphenyl-4,4′-dicarboxylic acid bis(4-aminophenyl)ester was contained in an amount of 1.33 g (Yield: 78% on the basis of biphenyl-4,4′-dicarboxylic acid bis(4-chlorophenyl)ester).

### Example C-8

### Synthesis of biphenyl-4,4′-dicarboxylic acid bis(4-aminophenyl)ester

In a flask having an inner volume of 25 ml and equipped with a stirring device, a thermometer and a dropping funnel, 1.85 g (4.00 mmol) of biphenyl-4,4′-dicarhoxylic acid bis(4-chlorophenyl)ester synthesized in Example C-5, 1.31 g (12 mmol) of 4-aminophenol, 15 ml of dimethyl sulfoxide and 0.122 g (0.800 mmol) of 1,8-diazabicyclo[5,4,0]-7-undecene were mixed under an argon gas stream, and the resulting mixture was stirred at a liquid temperature of 92° C. for 2.5 hours. After completion of the reaction, the resulting reaction solution was cooled to 25° C. and then 15 ml of water was added thereto, and the mixture was filtered. The resulting solid was washed with 15 ml of water and 10 ml of methanol in order, and then dried to obtain 1.67 g of a light yellow solid.

The light yellow solid was analyzed by high performance liquid chromatography and as a result, biphenyl-4,4′-dicarboxylic acid bis(4-aminophenyl)ester (desired product) and biphenyl-4,4′-dicarboxylic acid 4-(4-aminophenyl)ester 4′-(4-chlorophenyl)ester (precursor of the desired product) were prepared in the ratio of 94:6 (areal percentage). Furthermore, this solid was quantitatively analyzed by high performance liquid chromatography and as a result, biphenyl-4,4′-dicarboxylic acid bis(4-aminophenyl)ester was contained in an amount of 1.51 g (Yield: 89% on the basis of biphenyl-4,4′-dicarboxylic acid bis(4-chlorophenyl)ester).

### Example C-9

### Synthesis of biphenyl-4,4′-dicarboxylic acid bis(2,4-dichlorophenyl)ester

To a flask having an inner volume of 500 ml and equipped with a stirring device and a thermometer were added 10.4 g (103 mmol) of triethylamine, 170 ml of tetrahydrofuran and 12.9 g (78.8 mmol) of 2,4-dichlorophenol. To this mixed solution was added 10.0 g (35.8 mmol) of 4,4′-biphenyldicarbonyl chloride while maintaining the liquid temperature at 30° C. or less, and then the resulting mixture was stirred at 25° C. for 4.5 hours. After completion of the reaction, the reaction solution was filtered. The filtered residue was suspended in 333 ml of water and stirred at 25° C. for 1 hour This mixture was filtered again, and the resulting solid was washed with 400 ml of water and 40 ml of tetrahydrofuran in order and then dried to obtain 17.8 g (33.4 mmol) of biphenyl-4,4′-dicarboxylic acid bis(2,4-dichlorophenyl)ester as a white solid (Isolation yield: 93% on the basis of 4,4′-biphenyldicarbonyl chloride).

The resulting biphenyl-4,4′-dicarboxylic acid bis(2,4-dichlorophenyl)ester was a novel compound shown by the following physical properties.

¹H-NMR(300 MHz,THF-d₈,δ(ppm)); 7.38-7.50(4H,m),7.60-7.71(2H,m),7.93-8.01(4H,m),8.25-8.39(4H,m)

### Example C-10

### Synthesis of biphenyl-4,4′-dicarboxylic acid bis(4-aminophenyl)ester

In a flask having an inner volume of 25 ml and equipped with a stirring device and a thermometer, 2.13 g (4.00 mmol) of biphenyl-4,4′-dicarboxylic acid bis(2,4-dichlorophenyl)ester synthesized in Example C-9, 1.31 g (12.0 mmol) of 4-aminophenol, 15 ml of N,N-dimethylformamide and 0.122 g (0.800 mmol) of 1,8-diazabicyclo[5,4,0]-7-undecene were mixed under an argon gas stream. This mixed solution was heated and stirred at a liquid temperature of 92° C. for 16 hours. After completion of the reaction, the reaction solution was cooled to 25° C. and 15 ml of water was added thereto. This reaction solution was filtered, and the resulting solid was washed with 15 ml of water and 10 ml of methanol in order, and then dried to obtain 1.60 g of a light brown solid.

The light brown solid was analyzed by high performance liquid chromatography and as a result, the ratio of biphenyl-4,4′-dicarboxylic acid bis(4-aminophenyl)ester (desired product) to biphenyl-4,4′-dicarboxylic acid 4-(4-aminophenyl)ester 4′-(2,4-dichlorophenyl)ester (precursor of the desired product) was 99:1 (areal percentage). Furthermore, this solid was quantitatively analyzed by high performance liquid chromatography and as a result, the amount of biphenyl-4,4′-dicarboxylic acid bis(4-aminophenyl)ester was 1.06 g (Yield: 62% on the basis of biphenyl-4,4′-dicarboxylic acid bis(2,4-dichlorophenyl)ester).

### Synthesis Examples of Diamine Compounds according to Production Method II-2

Next, Synthesis Examples of the diamine compounds according to Production Method II-2 will be illustrated in detail.

### Example D-1

### (A) Synthesis of biphenyl carbamide compound (3,3′-(biphenyl-4,4′-dicarbonyl)-bis-1,3-thiazolidine -2-thione)

To a flask having an inner volume of 200 ml and equipped with a stirring device, a thermometer and a dropping funnel were added 7.26 g (0.072 mol) of triethylamine, 99 ml of tetrahydrofuran and 6.58 g (0.055 mol) of 2-thiazoline-2-thiol. Then, 7.00 g (0.025 mol) of 4,4′-biphenyldicarbonyl chloride was slowly added thereto while maintaining the liquid temperature at 10° C. and the resulting mixture was reacted at room temperature for 17 hours. After completion of the reaction, the reaction solution was filtered, and the filtered residue was suspended in 300 ml of water and stirred at 25° C. for 1 hour. The resulting solution was filtered and then the filtered residue was washed with 200 ml of water and 50 ml of tetrahydrofuran in order, and the resulting solid was then dried to obtain 10.18 g of 3,3′-(biphenyl-4,4′-dicarbonyl)-bis-1,3-thiazolidine-2-thione as yellow powder (Isolation yield: 92% on the basis of 4,4′-biphenyldicarbonyl chloride).

3,3′-(biphenyl-4,4′-dicarbonyl)-bis-1,3-thiazolidine-2 -thione was a novel compound shown by the following physical properties.

¹H-NMR(THF-d₆,δ(ppm)); 3.58-3.92(2H,m), 4.45-4.59(2H,m), 7.76-7.93(8H,m)

### Example D-2

### (B) Synthesis of biphenyl-4,4′-dicarboxylic acid bis(4-aminophenyl)ester

To a flask having an inner volume of 25 ml and equipped with a stirring device, a thermometer and a dropping funnel was added 0.35 g (8.8 mmol) of 60% sodium hydride, and then 14 ml of tetrahydrofuran was added in an argon atmosphere while the liquid temperature was maintained at 5° C. Subsequently, 0.87 g (8.0 mmol) of 4-aminophenol dissolved in 34 ml of tetrahydrofuran was slowly added dropwise thereto while maintaining the liquid temperature at 5° C., and then the resulting mixture was stirred at 25° C. for 20 minutes. Thereafter, 1.78 g (4.0 mmol) of the biphenyl carbamide compound (3,3′-(biphenyl-4,4′-dicarbonyl)-bis-1,3-thiazolidine-2-thione) synthesized in Example D-1 was slowly added dropwise to the mixed solution while maintaining the liquid temperature at 5° C., and the resulting mixture was reacted at the same temperature for 2 hours. After completion of the reaction, the reaction solution was filtered and divided into a filtered residue and a filtrate. The resulting filtered residue was washed with 10 ml of tetrahydrofuran and dried to obtain 1.30 g of a skin color solid This solid was analyzed by high performance liquid chromatography and as a result, biphenyl-4,4′-dicarboxylic acid bis(4-aminophenyl)ester was contained in an amount of 1.20 g (Yield: 71% on the basis of 3,3′-(biphenyl-4,4′-dicarbonyl) -bis-1,3-thiazolidine-2-thione).

On the other hand, the resulting filtrate was concentrated under reduced pressure. To 2.18 g of this concentrate, 10 ml of methanol was added, and the mixture was stirred at room temperature for 30 minutes and then further filtered. The resulting filtered residue was dried to obtain 0.40 g of a skin color solid. This solid was analyzed by high performance liquid chromatography and as a result, biphenyl-4,4′-dicarboxylic acid bis(4-aminophenyl)ester was contained in an amount of 0.34 g (Yield: 20% on the basis of 3,3′-(biphenyl-4,4′-dicarbonyl)-bis-1,3-thiazolidine-2-thione).

Herein, physical properties of biphenyl-4,4′-dicarboxylic acid bis(4-aminophenyl)ester were as follows.

¹H-NMR(DMSO-d₆,δ(ppm)); 5.10(4H,brs,NH2), 6.40-6.66(4H,m), 6.90-6.98(4H,m), 7.80-8.08(4H,m), 8.01-8.25(4H,m)

### Example D-3

### (B) Synthesis of biphenyl-4,4′-dicarboxylic acid bis(4-aminophenyl)ester

To a flask having an inner volume of 25 ml and equipped with a stirring device, a thermometer and a dropping funnel were added 0.77 g (8.0 mmol) of sodium t-butoxide and 14 ml of tetrahydrofuran. Then 0.87 g (8.0 mmol) of 4-aminophenol was slowly added dropwise thereto, and then the resulting mixture was stirred at 25° C. for 20 minutes. Thereafter, 1.78 g (4.0 mmol) of the biphenyl carbamide compound (3,3′-(biphenyl-4,4′-dicarbonyl)-bis-1,3-thiazolidine-2-thione) synthesized in Example D-1 was slowly added dropwise to the mixed solution while maintaining the liquid temperature at 5° C., and the resulting mixture was reacted at the same temperature for 2 hours. After completion of the reaction, 20 ml of methanol was added to the resulting reaction solution, and the mixture was stirred at room temperature for 30 minutes and then further filtered. The resulting filtered residue was dried to obtain 1.60 g of a skin color solid. This solid was analyzed by high performance liquid chromatography and as a result, biphenyl-4,4′-dicarboxylic acid bis(4-aminophenyl)ester was contained in an amount of 1.44 g (Yield; 85% on the basis of 3,3′-(biphenyl-4,4′-dicarbonyl)-bis-1,3-thiazolidine-2-thione).

### INDUSTRIAL APPLICABILITY

The polyimide of the present invention is excellent in heat resistance, has a low water absorption percentage and a low linear hygroscopic expansion coefficient, and is excellent in dimensional stability. Furthermore, the diamine compound of the formula (1) and its intermediate are useful as a raw material for the production of a polyimide.

1. A polyimide obtained by reacting a tetracarboxylic acid component with a diamine component containing a diamine compound represented by the following general formula (1): 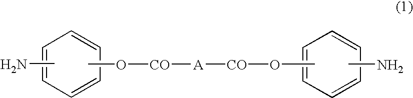
wherein, A represents a biphenylene group which may be substituted with an alkyl group having up to 4 carbon atoms.

2. The polyimide according to claim 1, wherein the tetracarboxylic acid component comprises 3,3′,4,4′-biphenyltetracarboxylic dianhydride in an amount of 10 mol % or more of all tetracarboxylic acid components.

3. The polyimide according to claim 1, wherein the diamine compound represented by the above general formula (1) comprises a compound represented by the following formula (1a):

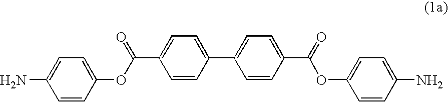

4. A polyimide film comprising the polyimide according to claim 1.

5. A diamine compound represented by the general formula (1): 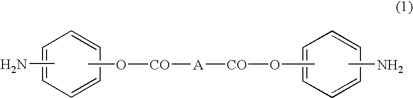
wherein, A represents a biphenylene group which may be substituted with an alkyl group having up to 4 carbon atoms.

6. Biphenyl-4,4′-dicarboxylic acid bis(4-aminophenyI)ester represented by the following formula (1a):

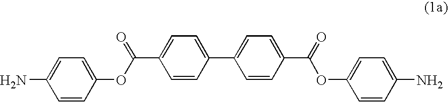

7. A method for producing the diamine compound represented by the general formula (1) according to claim 5, comprising:
reacting a biphenyl dicarbonyl halide derivative represented by the general formula (2): 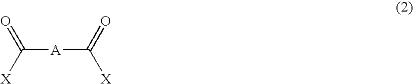
wherein, A represents a biphenylene group which may be substituted with an alkyl group having up to 4 carbon atoms; and X represents a halogen atom,
with nitrophenol in the presence of a base, whereby producing biphenyl-dicarboxylic acid bis(nitrophenyl)ester represented by the general formula (3): 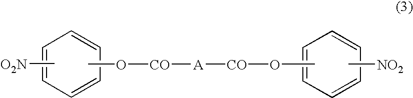
and
reducing biphenyl-dicarboxylic acid bis(nitrophenyl)ester represented by the above general formula (3).

8. A method for producing the diamine compound represented by the general formula (1) according to claim 5, comprising:
reacting a biphenyl carbonyl derivative represented by the general formula (21): 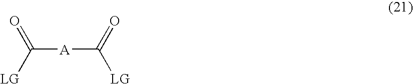
wherein, A has the same meaning as defined above; and LG is a leaving group which can be exchanged with an aminophenoxy group, with aminophenol in the presence of a base.

9. The method for producing the diamine compound according to claim 8, wherein the general formula (21) is a biphenyl-dicarboxylic acid bis(aryl)ester compound represented by the following general formula (22): 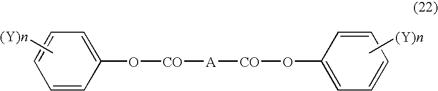
wherein, A has the same meaning as defined above; Y represents a halogen atom, a nitro group, a trifluoromethyl group, a cyano group or an acetyl group; and n represents an integer of 0 to 3.

10. The method for producing the diamine compound according to claim 9, wherein the biphenyl-4,4′-dicarboxylic acid bis(aryl)ester compound represented by the above general formula (22) is obtained by reacting the biphenyldicarbonyl halide derivative represented by the general formula (2): 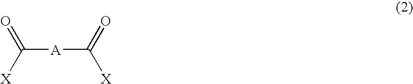
wherein, A represents a biphenylene group which may be substituted with an alkyl group having up to 4 carbon atoms; and X represents a halogen atom,
a hydroxyaryl compound represented by the general formula (23): 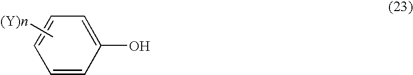
wherein, Y and n have the same meaning as defined above, and a base.

11. The method for producing the diamine compound according to claim 9, wherein the reaction is carried out without removing the generated hydroxyaryl compound from the reaction solution.

12. The method for producing the diamine compound according to claim 9, wherein the reaction is carried out while removing the generated hydroxyaryl compound from the reaction solution.

13. The method for producing the diamine compound according to claim 9, wherein a substitution position in the aryl moiety of the biphenyl-dicarboxylic acid bis(aryl)ester compound of the above general formula (22) is at least one substitution position selected from the group consisting of the 2 position, the 4 position and the 6 position.

14. The method for producing the diamine compound according to claim 9, wherein Y is a chlorine atom.

15. (canceled)

16. (canceled)

17. The method for producing the diamine compound according to claim 8, in which the above general formula (21) is a biphenyl carbamide compound represented by the general formula (32): 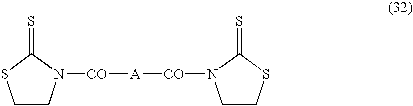
wherein, A has the same meaning as defined above.

18. The method for producing the diamine compound according to claim 17, wherein the biphenyl carbamide compound represented by the above general formula (32) is obtained by reacting the biphenyldicarbonyl halide derivative represented by the general formula (2): 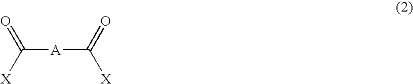
wherein, in the formula, A represents a biphenylene group which may be substituted with an alkyl group having up to 4 carbon atoms; and X represents a halogen atom,
2-thiazoline-2-thiol and a base.

19. (canceled)

20. (canceled)
